(19) United States
(12) Patent Application Publication
(10) Pub. No.: US 2018/0165818 A1
(43) Pub. Date: Jun. 14, 2018

(54) METHODS AND SYSTEMS FOR REGISTERING MULTIPLE DENTAL IMAGERIES

(71) Applicant: Ormco Corporation, Orange, CA (US)

(72) Inventors: Evan Yifeng Tsai, Rancho Cucamonga, CA (US); Ian Kitching, Rancho Cucamonga, CA (US)

(21) Appl. No.: 15/816,110

(22) Filed: Nov. 17, 2017

Related U.S. Application Data
(60) Provisional application No. 62/433,441, filed on Dec. 13, 2016.

(51) Int. Cl. G06T 7/33; A61C 7/00; A61C 9/00; A61B 6/03; A61B 6/14; A61B 6/00

(52) U.S. Cl. CPC G06T 7/337; A61C 7/002; A61C 9/0046; A61B 6/032; A61B 6/14; G06T 2207/30036; A61B 6/5247; G06T 2207/10116; G06T 2207/10081; G06T 2207/20221; A61B 6/4085

(57) ABSTRACT

A method for registering imageries of a tooth includes integrating imageries to roughly register two imageries. The method includes cross-sectioning the imageries through the tooth and moving one imagery relative to another one. Moving includes at least one of rotating and translating an imagery and may be relative to a characteristic feature of the tooth in the imagery. The tooth may be an anterior tooth, such as an incisor. Cross-sectioning may be at the characteristic feature in a sagittal plane, a transverse plane, and a coronal plane. And, moving may include at least one of rotating and translating the second imagery on a plane-by-plane basis in each plane. The method may include evaluating an error in the position of the imageries and repeating moving the imageries. A dental registration system includes a first imaging system and a second imaging system to capture different imageries of the teeth.

### CROSS REFERENCE TO RELATED APPLICATIONS

This application claims the benefit of U.S. Provisional Patent Application Ser. No. 62/433,441 filed on Dec. 13, 2016, the disclosure of which is expressly incorporated by reference herein in its entirety.

### TECHNICAL FIELD

The invention generally relates to methods and to systems for orthodontic treatment planning and, in particular, to methods and systems for registering multiple imageries of a patient's teeth.

### BACKGROUND

Orthodontics is a specialty of dentistry that is concerned with improvement of the appearance of a patient's teeth, including correction of malocclusions and other defects. Orthodontic appliances, such as orthodontic brackets and other devices, are often attached to a patient's teeth. These devices apply forces to the teeth to move them into their orthodontically correct orientations according to a treatment plan. The treatment plan may be developed based on modeling of the patient's teeth.

To that end, a practitioner may initially prepare a digital model. This may include taking impressions of the patient's teeth and then capturing images of those impressions. Instead of impressions, the practitioner may capture photographic imagery or capture x-ray imagery of the patient's teeth and the surrounding skeletal structure. The information contained in the captured imagery is then used to create the digital model of one or more of the patient's teeth. Digital models may include both two-dimensional and three-dimensional models. The practitioner may then virtually manipulate the digital models in the development of the treatment plan for that patient.

By way of example, models may be developed with an intra-oral imaging system that allows a practitioner to see the inside of a patient's mouth and display the topographical characteristics of teeth on a display monitor. Certain intra-oral imagers may include an intra-oral camera with a light source. The intra-oral imager may be fabricated in the form of a slender wand that may be approximately the size of a dental mirror with the camera and light source at or near one end. The camera may be capable of magnifying captured images by 40 times or more. The wand may be inserted into the oral cavity so that the practitioner may capture images of the teeth and the gingiva. Magnifying the images allows the practitioner to visually inspect the teeth to detect defects. The information captured by the intra-oral camera may be displayed on a monitor and may be transmitted to a computational device.

As another example, the practitioner may use data collected via cone beam computed tomography (CBCT), which involves the use of a rotating CBCT scanner, to collect data regarding the teeth, surrounding bone structure, and soft tissue. The data may be transmitted to a computational device to generate CBCT imagery. The data may include three-dimensional anatomical data that can be manipulated and visualized with specialized software. The captured information may provide the practitioner with an accurate reproduction of the topographical characteristics and arrangement of the crowns of teeth in one or both of the patient's maxillary or mandible jaws. Either or both types of digital data may be used to develop the treatment plan and/or may be used to assess orthodontic treatment progress.

Plan development may also include customized appliances. Treatment may therefore include identification and design of orthodontic appliances specific to the patient's condition. The effect of the appliance may be modeled in conjunction with the digital imagery prior to placement on the patient's teeth. Software platforms allow the practitioner to digitally manipulate the model. In this way, the targeted final positions of the teeth and the treatment plan to obtain those final positions with the appliances may be refined prior to actual treatment of the patient. Refinement of treatment may include further customization of the orthodontic appliances to a particular patient. The customized appliances may then be manufactured according to the treatment plan specified.

After initiation of the treatment, treatment progress may be monitored and refined via the digital model. For example, the treatment efficacy may be monitored and adjusted as determined in conjunction with digital models of the patient's teeth. Periodic adjustments are often needed for satisfactory completion of treatment.

In view of the above, it is advantageous to capture accurate information regarding each tooth in the patient's jaws. It will be appreciated that the more accurate a model is, the more effective the orthodontic treatment is likely to be. However, there are problems with the accuracy of some types of information. The resulting imagery is then less accurate than desired. For example, there are problems with capturing accurate images of the patient's teeth, particularly data indicative of the location and orientation of the roots of each tooth. Because the roots are buried in tissue, they cannot be seen with visible light, which is typically used to capture intra-oral imagery of the teeth. This leaves locating the roots with x-rays or similar technologies.

As is known, x-rays are capable of differentiating soft tissue from bone and so can provide information as to the location and orientation of the roots of each of the patient's teeth. There are, however, some problems with x-rays. For one, x-rays are capable of damaging human tissue. Thus, when x-rays are used, the patient is only exposed to the lowest dosage necessary to obtain the information for treatment. While being safer for the patient, as the dosage decreases, the information obtained from the x-rays becomes less accurate. There is therefore a trade-off between dosage and model accuracy. As a consequence, because patient safety is paramount, low dosage is used such that the location and orientation of the roots are not generally accurately known. More often than not, images formed from x-ray data are fuzzy and unclear at low dose x-ray levels. The presence of metal fillings and such in or near a patient's teeth only serves to obscure and distort imagery that may already be fuzzy. While there are methods to overcome image distortion due to metal fillings, these methods most often require significant data pre-processing. This type of pre-processing requires significant operator involvement and/or very sophisticated algorithms and for that reason, pre-processing is avoided in all but the most severe cases, which may justify the added time and expense.

Thus, while creation of dental models has been generally successful, orthodontists, software and device manufacturers continually strive to improve the accuracy of models, particularly those that are based on x-ray data, to afford the most efficient and cost effective treatment possible while using only the lowest x-ray dosage and avoiding problems associated with metal fillings and such. In this regard, there exists a need for methods and systems for registration of imagery from multiple sources to form accurate digital models.

### SUMMARY

The present invention addresses the foregoing and other shortcomings and drawbacks of dental model accuracy heretofore known for use in orthodontic treatment. While the invention will be described in connection with certain embodiments, it will be understood that the invention is not limited to these embodiments. On the contrary, the invention includes all alternatives, modifications, and equivalents as may be included within the spirit and scope of the present invention.

In accordance with the principles of the present invention, a method for registering imageries of a patient's tooth, such as a first imagery of a patient's tooth and a second imagery of the patient's tooth, comprises integrating the first imagery of a tooth and the second imagery of the tooth so that the first imagery is roughly registered with the second imagery. The method includes cross-sectioning the first imagery and the second imagery through the tooth, and moving the second imagery relative to the first imagery to align the cross section of the second imagery with the cross section of the first imagery.

In one embodiment, moving includes at least one of rotating and translating the second imagery relative to the first imagery. Rotating and translating may be relative to a characteristic feature of the tooth in the first imagery.

In one embodiment, the tooth is an anterior tooth, such as an incisor.

In one embodiment, the method further includes selecting a characteristic feature on the tooth and wherein cross-sectioning includes cross-sectioning the first imagery at the characteristic feature.

In one embodiment, selecting a characteristic feature of the tooth includes selecting a characteristic feature of an incisor.

In one embodiment, cross-sectioning includes cross-sectioning along at least one of a sagittal plane, a transverse plane, and a coronal plane.

In one embodiment, moving includes at least one of rotating and translating the second imagery on a plane-by-plane basis in each of the sagittal plane, the transverse plane, and the coronal plane.

In one embodiment, after moving the second imagery, the method further includes evaluating an error in the position of the second imagery relative to the first imagery.

In one embodiment, the method further includes repeating moving the second imagery relative to the first imagery to further align the cross section of the second imagery with the cross section of first imagery.

In one embodiment, after moving the second imagery, the method further includes moving the first imagery relative to the second imagery.

In one embodiment, the first imagery is a surface imagery and the second imagery is a volumetric imagery.

According to one aspect of the invention, there is a method for registering a first imagery of a patient's tooth and a second imagery of the patient's tooth. The method comprises integrating the first imagery of a tooth and the second imagery of the tooth so that the first imagery is roughly registered with the second imagery. The method further includes selecting a characteristic feature of the tooth in the first imagery and moving the second imagery relative to the first imagery about the characteristic feature so as to align the second imagery with the first imagery.

In one embodiment, selecting the characteristic feature includes selecting a feature on an anterior tooth. Selecting the characteristic feature may include selecting a feature on an incisor.

In one embodiment, the method further includes cross-sectioning the first imagery through at least the characteristic feature.

In one embodiment, cross-sectioning includes cross-sectioning along at least one of a sagittal plane, a transverse plane, and a coronal plane.

In one embodiment, cross-sectioning includes cross-sectioning the second imagery.

In one embodiment, the first imagery is a surface imagery of the tooth and the second imagery is a volumetric imagery of the tooth.

In one embodiment, moving includes at least one of rotating and translating the second imagery.

In one embodiment, after moving the second imagery, the method further includes evaluating an error in a position of the second imagery relative to the first imagery.

In one embodiment, the method further includes repeating moving the second imagery relative to the first imagery.

In one embodiment, after moving the second imagery, the method further includes moving the first imagery relative to the second imagery about the characteristic feature.

In one embodiment, the first imagery is a surface imagery and the second imagery is a volumetric imagery.

According to one aspect of the invention, there is a dental registration system that comprises a first imaging system that is capable of capturing information that is usable to prepare a first imagery of a patient's tooth. The dental registration system further includes a second imaging system that is capable of capturing information that is usable to prepare a second imagery of the patient's tooth. The second imagery is different than the first imagery. The dental registration system further includes at least one computer that is operatively coupled to the first imaging system and the second imaging system and includes an integrating application that is capable of manipulating the first imagery and the second imagery. The integrating application is capable of cross-sectioning the first imagery and the second imagery along at least one of a sagittal plane, a transverse plane, and a coronal plane and is capable of moving the second imagery relative to the first imagery.

### BRIEF DESCRIPTION OF THE DRAWINGS

The accompanying drawings, that are incorporated in and constitute a part of this specification, illustrate various embodiments of the invention and, together with the detailed description of the embodiments given below, serve to explain the embodiments of the invention.

FIG. 1 is a schematic of an orthodontic imagery registration system according to one embodiment of the invention;

FIG. 2 is an exemplary intra-oral imagery (i.e., surface imagery) of a patient's mandibular and maxillary arches;

FIGS. 3, 4, and 5 are transverse, sagittal, and coronal cross-sectional imageries, respectively, of an exemplary CBCT image (i.e., volumetric imagery) of the patient's maxillary jaw;

FIG. 6 is a minimum slice view according to one embodiment of the invention taken in a transverse cross-sectional view of the maxillary jaw of the exemplary volumetric imagery shown in FIGS. 3, 4, and 5;

FIG. 7 is a maximum slice view according to one embodiment of the invention taken in a transverse cross-sectional view of the maxillary jaw of the exemplary volumetric imagery shown in FIGS. 3, 4, and 5;

FIG. 8 is a model slice view according to one embodiment of the invention taken in a transverse cross-sectional view of the maxillary jaw of the exemplary volumetric imagery shown in FIGS. 3, 4, and 5;

FIGS. 9A, 9B, and 9C depict an exemplary edge finding method beginning at the model slice view shown in FIG. 8;

FIG. 10 is a flow chart according to one embodiment of the invention;

FIGS. 11, 12, and 13 illustrate an initial registration at an incisor of the imagery shown in FIG. 2 and the same incisor in a slice of a volumetric imagery shown in FIG. 8 according to one embodiment of the invention;

FIG. 14 is a sagittal plane cross section taken through the incisor of the overlapping imagery of the maxillary arch shown in FIG. 2 and a volumetric imagery from which FIGS. 6, 7, and 8 were taken according to one embodiment of the invention;

FIG. 15 is the sagittal cross section shown in FIG. 14 following rotation and translation of the volumetric imagery toward the surface imagery according to one embodiment of the invention;

FIG. 16 is a schematic representation of a central incisor;

FIG. 17 is a transverse plane cross section taken through the incisor of the overlapping surface imagery of the maxillary arch shown in FIG. 2 and a volumetric imagery from which FIGS. 6, 7, and 8 were taken according to one embodiment of the invention;

FIG. 18 is an imagery of the transverse cross section shown in FIG. 17 following rotation and translation of the volumetric imagery toward the surface imagery according to one embodiment of the invention;

FIGS. 19A and 19B are a coronal cross section and a sagittal cross section, respectively, of the overlapping imagery as shown in FIGS. 11, 12, and 13 illustrating alignment between molars prior to rotation and translation of the surface imagery and the volumetric imagery;

FIGS. 20A and 20B are imagery shown in FIGS. 19A and 19B following rotation and translation of the coronal cross section of FIG. 19A of the volumetric imagery toward the surface imagery and the result of that movement on the sagittal cross section of FIG. 19B according to one embodiment of the invention;

FIGS. 21A and 21B are imagery of a sagittal cross section and a coronal cross section, respectively, of the overlapping imagery as shown in FIGS. 11, 12, and 13 illustrating alignment between molars prior to rotation and translation of the surface imagery and the volumetric imagery;

FIGS. 22A and 22B are imagery shown in FIGS. 21A and 21B following rotation and translation of the sagittal cross section of FIG. 21A of the volumetric imagery toward the surface imagery and the result of that movement on the coronal cross section of FIG. 21B according to one embodiment of the invention; and

FIG. 23 is a schematic view in which a pair of incisors is rotated and translated according to one embodiment of the invention.

### DETAILED DESCRIPTION

To these and other ends, embodiments of the present invention include systems, methods, and computer readable storage mediums that are capable of forming an accurate model of a patient's teeth through registration of multiple independent imageries. In general, highly accurate surface data and/or shape data of the crowns of a patient's teeth, such as from intra-oral scanning or other scanned data, may be registered with less accurate, though more comprehensive data, such as volumetric data acquired from cone beam computed tomography (CBCT). This integration may ultimately provide a more accurate model of the patient's teeth even when low x-ray dosage x-rays are used and/or when the patient has one or more fillings that tend to distort x-rays. Thus, registration according to embodiments of the invention may produce data from a combination of data from one type of imagery that includes portions of one or more teeth together with data from another type of imagery in which the portions of the patient's teeth are not visible. Imagery may include any information related to the patient's teeth, including, but not limited to, data indicative of any single one or a combination of the surface, shape, relative location, and orientation of one or more of the patient's teeth. Imagery may be displayed for the clinician's visual assessment and manipulation.

Following registration, the model of the patient's teeth may be a compilation of identifiable features found in the separate imageries. For example, all or a portion of the crowns depicted in the model may be from one imagery and all or a portion of the roots depicted in the model may be from a separate imagery. Registration may include independent alignment of the imageries in separate predetermined cross-sectional planes. The predetermined planes may be taken through an overlay of the separate imageries at a common tooth feature. The common feature may include one or more landmarks characteristic to a specific tooth. The imageries may be manipulated or moved relative to one another about the common feature to bring about their registration.

Manipulation may include translating and rotating of one imagery relative to another imagery on a plane-by-plane basis. In each selected plane, a practitioner may manipulate the imageries to pattern match cross sections of a tooth in each of the imageries. Pattern matching may be based on human perception or an automatic algorithm and may be focused around anterior incisors. Embodiments of the invention therefore minimize the effect of distortion associated with fillings typically found in the crowns of posterior teeth. By embodiments of the invention, the accuracy of a digital model of the patient's teeth, particularly the roots of the patient's teeth, is improved. Registration according to embodiments of the invention improves the overall accuracy and detail of the constructed, final 3-D model of the patient's teeth. Advantageously, embodiments of the invention may not require best-fit calculations or threshold calculations and so lack the complication and error associated with those calculations. Nevertheless, embodiments of the invention produce accurate 3-D models of a patient's teeth because the methods and systems according to the present invention are less influenced by noisy data, distortion in the data, and/or incomplete data.

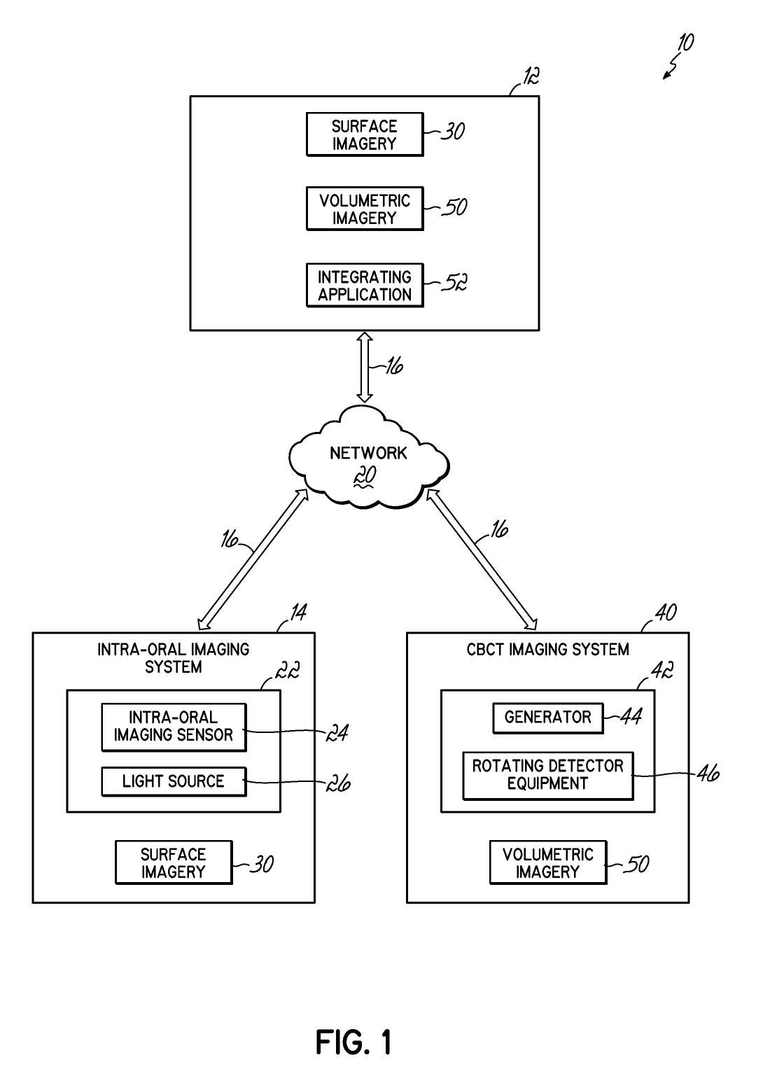

In an exemplary embodiment and with reference to FIG. 1, an orthodontic imagery registration system 10 includes a computer 12 and at least one imaging system 14. The computer 12 may be used to register multiple imageries from imaging system 14 and/or another imaging system. Imagery may include surface imagery of one or more of the patient's teeth. The computer 12 may include any suitable computational device, such as a personal computer, a server computer, a mini computer, a mainframe computer, a blade computer, a tablet computer, a touchscreen computing device, a telephonic device, a cell phone, a mobile computational device, dental equipment having a processor, etc. In certain embodiments, the computer 12 may provide web services or cloud computing services. More than one computer may also be used for storing data or performing the operations performed by the computer 12 in accordance with the embodiments of the invention. In the embodiment shown, while not being limited to any particular network, the computer 12 is operatively coupled via one or more wired or wireless connections 16 to the imaging system 14 over a network 20. The network 20 may comprise a local area network, the Internet, an intranet, a storage area network, or any other suitable network.

With continued reference to FIG. 1, in one embodiment, the imaging system 14 may capture surface data and/or shape data of one or more crowns of the patient's teeth, for example, via scanning an impression or a plaster model of the patient's teeth or by scanning the patient's teeth directly via an intra-oral scanner. In one embodiment, the imaging system 14 includes an intra-oral imaging system. Data of the crowns of the patient's teeth may be captured via the imaging system, such as by the intra-oral imaging system. As is known, intra-oral imaging systems may include a source of light and a detector, such as a camera, capable of detecting light after the light impinges upon and reflects from the surfaces of the patient's teeth. Intra-oral imaging systems may produce light in the visible light spectrum (i.e., 390 nm to 700 nm) which is capable of producing highly accurate surface data of the patient's teeth and gums.

With regard to an intra-oral imaging system, imaging system 14 may include a wand 22 having an imaging sensor 24 and a light source 26. The imaging sensor 24 may be an intra-oral camera that captures information regarding the oral cavity of the patient when exposed to light, for example from the light source 26. As is known, the practitioner may insert at least a portion of the wand 22 into the patient's mouth. Using the light source 26 and the imaging sensor 24, the practitioner may capture data of all or selected crowns of the patient's teeth. A crown of a tooth is a solid object, and the surfaces of the crown are boundaries of that object. The data collected may include surface data and/or shape data of the crown that may be represented by a surface mesh of node points connected by triangles, quadrilaterals, or another polygon mesh. The process of creating a mesh may be referred to as tessellation. The data collected may be used to construct imagery of one or more of the patient's teeth. The surface data may also include or be processed to extract information regarding the reflectivity and translucency of each of the patient's teeth. This information with regard to each crown is different from the reflectivity and translucency of the patient's gingiva. The imaging system 14 and/or the computer 12 may process the surface data in a manner so that the patient's crowns may be distinguished from the surrounding gingiva based on the reflectivity and the translucency differences. This may facilitate segmentation of the crown, when necessary. It will be appreciated that the imaging system 14 is not limited to an intra-oral imagining system.

As an alternative, the light source 26 may be incorporated into a scanner (not shown) that emits laser light. In this exemplary embodiment, the imaging sensor 24 may capture laser light reflected from an impression (e.g., a PVS impression) of the patient's teeth external to the patient. In this way, surface data for at least the crowns of the patient's teeth may be captured though the light source 26 and imaging sensor 24 may not be a part of an intra-oral camera.

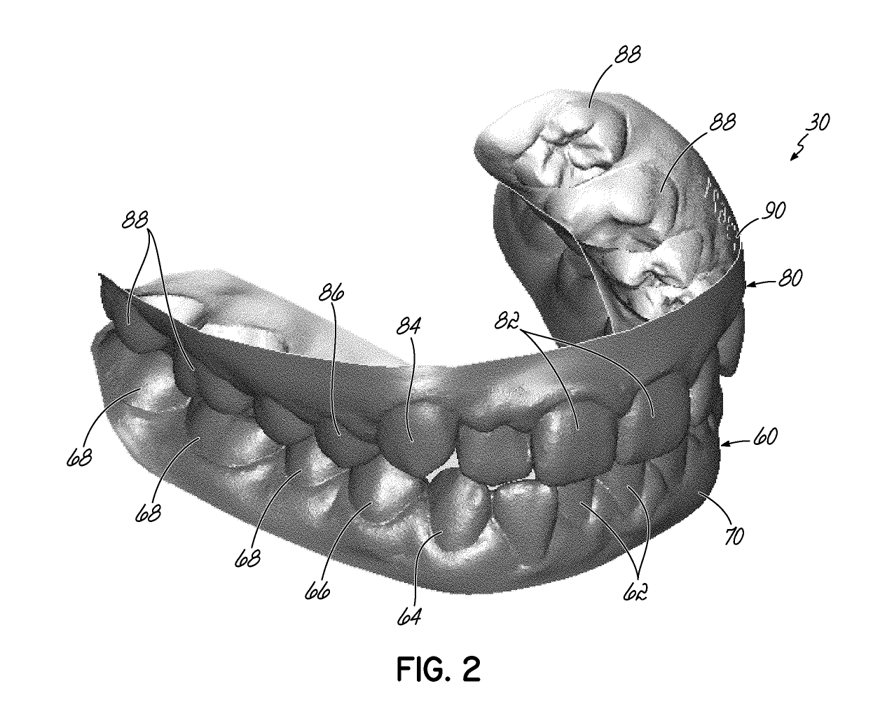

The imaging system 14 may ultimately construct surface imagery 30 (e.g., shown in FIG. 2) of the teeth in one or both of the patient's jaws from the surface data captured by the imaging sensor 24 or from another source. It will be appreciated that the surface imagery 30 constructed may include surface data from each of the crowns in both the maxillary and mandible jaws and surrounding gingiva. The surface imagery 30 may itself be a collection of separate images constructed from information captured by the imaging sensor 24 and later compiled by the imaging system 14. For example, the surface imagery 30 may be a compilation of separate images of the patient's maxillary and mandibular jaws. The surface imagery 30 may then be transferred to the computer 12, such as via the network 20, to be manipulated as is described below.

The imaging system 14 may also include another imaging system that is capable of producing imagery based on information that is different from the reflected light information used to construct surface imagery 30. To ease description only, in one embodiment, the computer 12 may also be operatively coupled via the wireless connection 16 to a cone beam computed tomography (CBCT) imaging system 40. While being described separately, the intra-oral imaging system 14 and the CBCT imaging system 40 may form a single system. The CBCT imaging system 40 may utilize the x-ray spectrum (i.e., wavelengths in the range of 0.01 nm to 10 nm) to produce images of the patient's teeth that are not readily observed with light in the spectrum of the intra-oral imaging system 14. Because x-rays pass through human tissue, x-rays may be utilized to develop imagery of the position and orientation of the roots of one or more of the patient's teeth. It will be appreciated that the methods and systems described herein are not limited to using visible light or x-rays. In fact, imagery produced from light at different wavelengths may be useful according to embodiments of the invention.

The exemplary CBCT imaging system 40 may include a rotating gantry 42. An x-ray generator 44 and an x-ray detector 46 may be affixed to and rotate with the gantry 42. The x-ray generator 44 may deliver a divergent pyramidal or cone-shaped source of ionizing radiation toward a patient positioned between the generator 44 and the x-ray detector 46. The x-rays detected by the detector 46 thus contain information specific to the skeletal and other tissues of the patient. Exemplary CBCT imaging systems include iCat from Imaging Sciences International, Carestream from Carestream Health, Planmeca from Planmeca USA, Inc., and Sirona from Dentsply Sirona.

As is known, x-rays pass through human tissue but are absorbed at different rates by different tissue. Because of the relatively high energy associated with x-rays, the practitioner may operate the CBCT imaging system 40 to deliver the lowest dose of radiation possible to the patient while the detector 46 captures sufficient volumetric data to develop a volumetric image by which embodiments of the invention are utilized to prepare a 3-D model of the patient's teeth. By way of example and not limitation, low radiation dosage means from about 1 μSv to about 30 μSv per scan. Low radiation dosages, while safer for the patient, have a downside. As the radiation dosage is reduced, the images of the patient's tissues are not as clean, that is, the detector 46 may be incapable of clearly differentiating the desired information from useless noise. In many instances, the level of noise relative to the useful data makes evaluation and treatment of the patient more difficult. Advantageously, embodiments of the invention may allow a lower dosage of x-rays in the above range to be used while maintaining or improving the accuracy of the 3-D model of the patient's teeth.

According to the CBCT imaging system 40, x-rays pass through the patient and into the detector 46 as the gantry 42 rotates around the patient. In a single rotation of the gantry 42, the CBCT imaging system 40 may generate volumetric data sufficient to form a plurality of planar projection images (from 100 to more than 600 image slices) of the patient's jaws and maxillofacial structures including soft tissue, hard tissue, teeth, etc. The individual planar projection images may be stacked to obtain volumetric imagery 50 of at least the patient's jaws. Stacking or reconstruction of the planar projection imagery by the CBCT imaging system 40 or another computer system (e.g., the computer 12) produces the volumetric imagery 50 that may have a voxel resolution ranging from about 0.4 mm to about 0.0076 mm. The volumetric imagery 50 may thus include volumetric data of both the crown and the root of one or more teeth in each of the patient's jaws as well as information regarding the surrounding tissues and contain data in three-dimensions of the patient's jaw. In one embodiment, the volumetric imagery 50 includes data regarding the location and orientation of each tooth in the patient's jaws in three dimensions. The volumetric imagery 50 obtained via x-rays may include radio density information. This information may be processed in combination with the surface data and/or shape data when registering the surface imagery 30 and the volumetric imagery 50, as is described below. Other light sources or other types of energy may be utilized to provide volumetric data regarding one or more of the patient's teeth. By way of example only, tomographic imagery, ultrasonic imagery, magnetic resonance imagery (MRI), etc., may be utilized to acquire volumetric data useful according to embodiments of the present invention.

Further in this regard, the exemplary embodiment shown in FIG. 1 includes two imaging systems, one providing surface imagery (i.e., intra-oral imaging system 14) and the other providing volumetric imagery (i.e., CBCT imaging system 40). According to embodiments of the invention, the corresponding surface imagery 30 and volumetric imagery 50 may then be received by and then manipulated within the computer 12. By way of example only, the computer 12 may include an integrating application 52 that is capable of simultaneously manipulating the surface imagery 30 and the volumetric imagery 50. The integrating application 52 may be implemented in certain embodiments in software, hardware, firmware or any combination thereof. By way of example only, and not limitation, the integrating application 52 may be written in Visual C++ and be operating on a Windows OS.

In general, integrating application 52 may include program code that typically includes computer readable instructions. These are resident at various times in various memory and storage devices in the computer 12 and that, when read and executed by one or more processors in the computer 12, cause that computer 12 to execute operations and/or elements embodying the various aspects of the embodiments of the invention. Computer readable program instructions for carrying out operations of the embodiments of the invention may be, for example, assembly language or either source code or object code written in any combination of one or more programming languages. Any particular program nomenclature herein is used merely for convenience, and thus embodiments of the invention are not limited to use solely in any specific application identified and/or implied by such nomenclature. Furthermore, given the generally endless number of manners in which computer programs may be organized into routines, procedures, methods, modules, objects, and the like, as well as the various manners in which program functionality may be allocated among various software layers that are resident within a typical computer (e.g., operating systems, libraries, API's, applications, applets, etc.), the embodiments of the invention are not limited to the specific organization and allocation of program functionality that may be described herein.

In one or more additional embodiments, the surface imagery 30 and the volumetric imagery 50 (or the corresponding data) may be stored in a storage medium (e.g., a disk drive, a floppy drive, a pen drive, a solid state device, an optical drive, etc.), and the storage medium may be coupled to the computer 12 for reading and processing by the integrating application 52. Computer readable storage media, which is inherently non-transitory, may include volatile and non-volatile and removable and non-removable tangible media implemented in any method or technology for storage of information, such as computer-readable instructions, data structures, program modules, or other data. Computer readable storage media may further include RAM, ROM, erasable programmable read-only memory (EPROM), electrically erasable programmable read-only memory (EEPROM), flash memory or other solid state memory technology, portable compact disc read-only memory (CD-ROM), or other optical storage, magnetic cassettes, magnetic tape, magnetic disk storage or other magnetic storage devices, or any other medium that can be used to store the desired information and which can be read by a computer. A computer readable storage medium should not be construed as transitory signals per se (e.g., radio waves or other propagating electromagnetic waves, electromagnetic waves propagating through a transmission media such as a waveguide, or electrical signals transmitted through a wire).

With reference to FIG. 2, and without being limited thereto, the exemplary surface imagery 30 is from an intra-oral imaging system, such as the intra-oral imaging system 14 shown in FIG. 1 and described above. As shown, the surface imagery 30 includes a mandibular jaw 60 composed of numerous teeth including the crowns of incisors 62, cuspids 64, bicuspids 66, and molars 68. The crowns of the teeth 62, 64, 66, 68 are shown projecting from gingiva 70. The surface imagery 30 shown in FIG. 2 is an un-segmented mesh surface. That is, the surface and/or shape data has not undergone preprocessing to remove the gingiva 70. Embodiments of the invention are not limited to use of un-segmented imagery.

Also shown in FIG. 2, the surface imagery 30 may further include a maxillary jaw 80 composed of images of numerous teeth including incisors 82, cuspids 84, bicuspids 86, and molars 88 projecting from gingiva 90 of the patient. The data of the mandibular jaw 60 may be captured separately from the data of the maxillary jaw 80. The data from each jaw 60 and 80 as captured by the intra-oral imaging system 14 may later be manipulated and combined via the intra-oral imaging system 14 or the integrating application 52, shown in FIG. 2 to form surface imagery 30. The surface imagery 30 differs from the volumetric imagery 50.

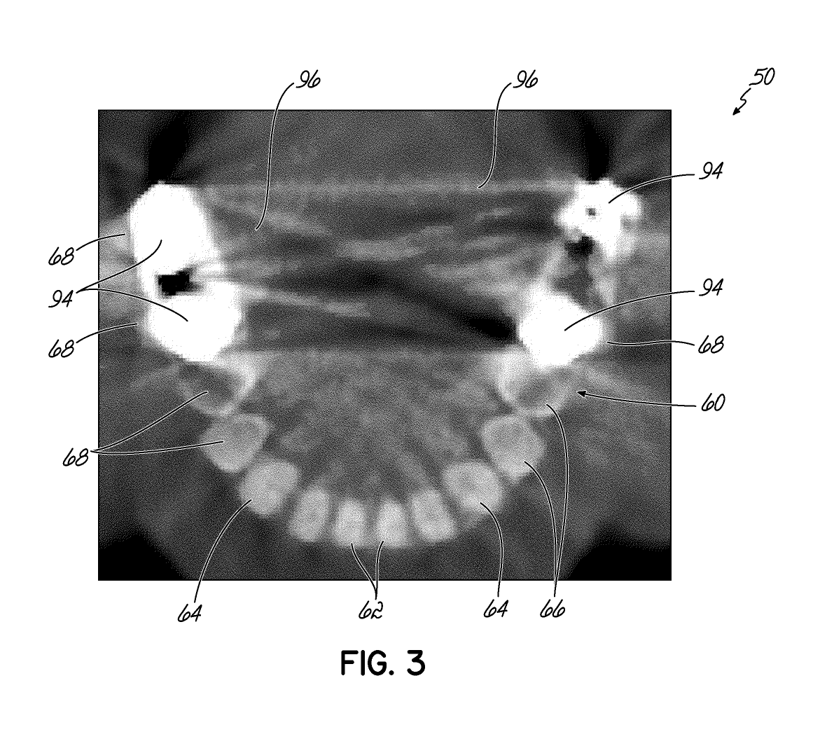

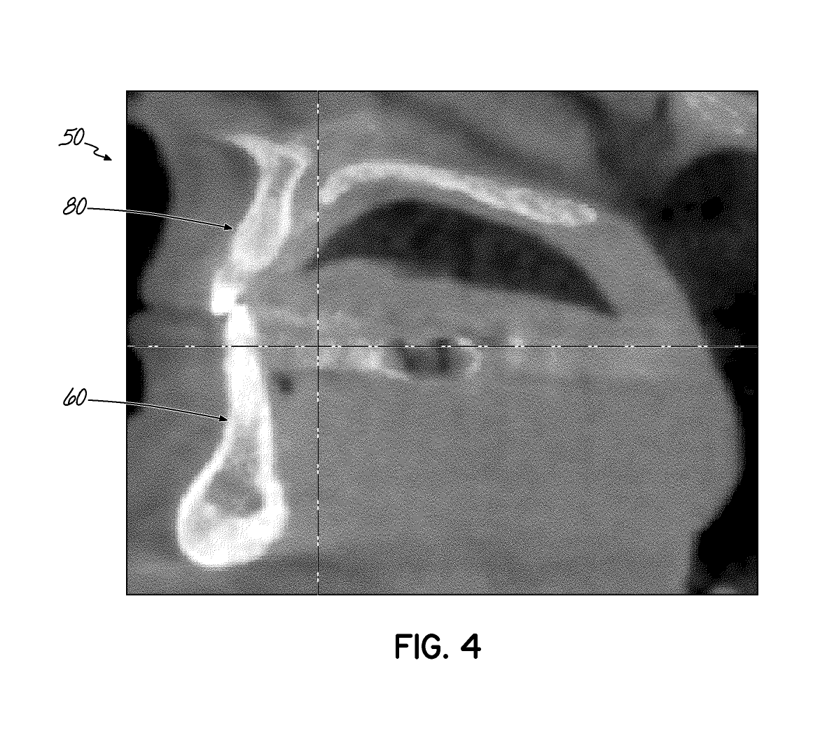

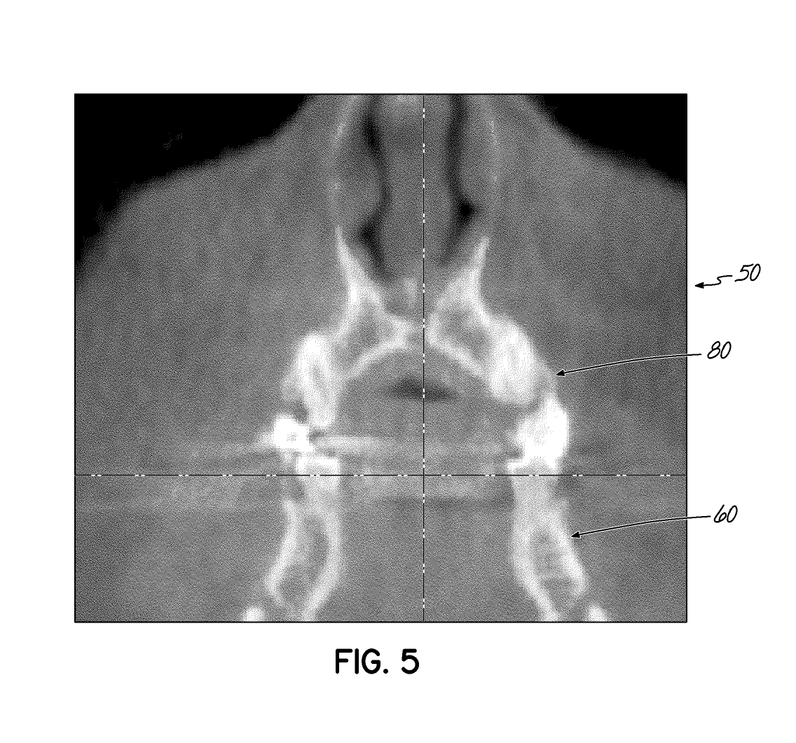

In that regard, the volumetric imagery 50 differs from the surface imagery 30 in at least the information about the same teeth of the patient. For example, as described above, the system 10 may include a CBCT imaging system 40 that produces volumetric imagery 50 with regard to the patient's teeth. In the exemplary embodiment of the volumetric imagery 50, specific cross sections of the imagery corresponding to the volumetric data are shown in FIGS. 3, 4, and 5. For example, in FIG. 3, a transverse plane cross section of the volumetric imagery 50 is shown; in FIG. 4, a sagittal plane cross section of the volumetric imagery 50 is shown; and in FIG. 5, a coronal cross section of the volumetric imagery 50 is shown.

Referring to FIG. 3, the volumetric imagery 50 also includes information on numerous teeth in the patient's mandibular jaw 60. In the cross section of the volumetric imagery 50, images of teeth 62, 64, 66, and 68 are shown. These are images of the same teeth of the patient shown in the surface imagery 30 of FIG. 2. However, in FIG. 3, data representing the roots of the teeth is available and so the roots are also visible in the imagery 30 and are shown best in the sagittal and coronal cross sections shown in FIGS. 4 and 5, respectively. The volumetric imagery 50 therefore provides information on the orientation of the teeth, including the corresponding roots of the patient's teeth, that is not available in the surface imagery 30 of FIG. 2.

In FIG. 3, it is also apparent that the patient has numerous fillings 94. The fillings 94, shown as bright splotches, cause significant noise in the volumetric imagery 50. Not only do the fillings 94 obscure information regarding the specific tooth which contains the filling, the fillings distort a region of x-ray information around the fillings 94. For example, distortions visually appear as streaks 96 that generally converge at the fillings 94.

In addition to the streaks 96, the data associated with the tooth 66 adjacent the tooth 68 with the filling 94 may be distorted. So, distortion in the data caused by fillings is not localized to a single tooth, i.e., the tooth with the filling, but can extend to one or more unfilled teeth adjacent the filling. Ultimately, in regions proximate (within one or two teeth) the filling 94, there may be no data or insufficiently accurate data with which to construct an accurate enough 3-D model on which to plan the patient's treatment. In this regard, the orientation of a tooth (particularly a crown) with a filling may remain unknown in the volumetric imagery 50 and thus present a problem for the practitioner for establishing a prognosis and for planning treatment based solely on the volumetric imagery 50.

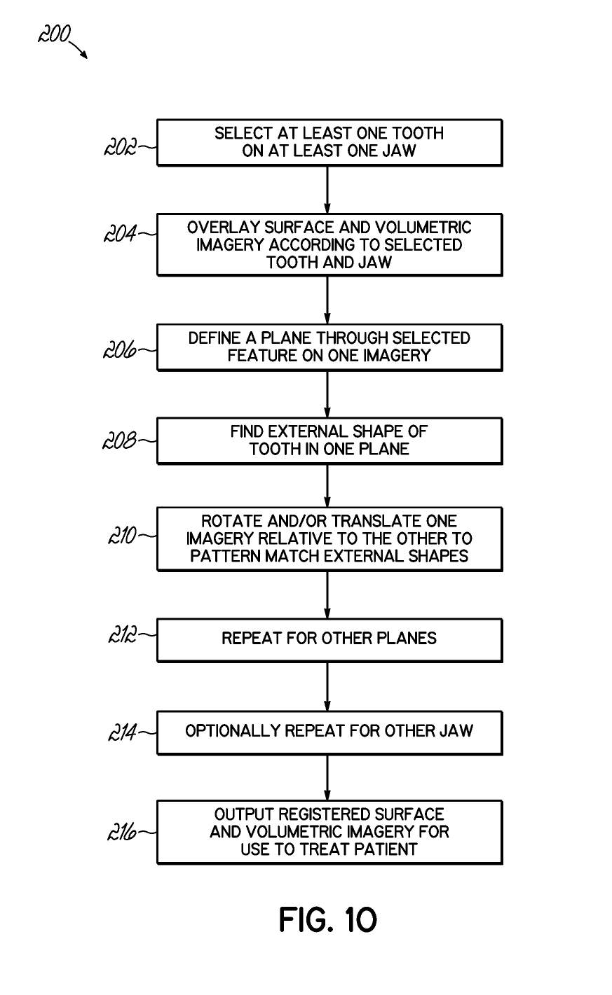

By contrast, in FIG. 3, the volumetric data of the incisors 62 is clear so as to be easily identifiable in the volumetric imagery 50, because these teeth are removed from the distorted region. It is unusual for a patient to have fillings in their incisors or in adjacent anterior teeth. Accordingly, a practitioner may select an incisor 62 or 82 about which the surface imagery 30 and the volumetric imagery 50 are registered, as is described below. By this selection, the distorted data is avoided. This is described below in one embodiment of the method of registration 200 at 202 with reference generally to FIG. 10. In that regard, prior to or during registration of the surface imagery 30 and the volumetric imagery 50, a practitioner may select one or more teeth remote from any fillings or other distortions of the volumetric imagery 50 for registration with identical teeth in the surface imagery 30.

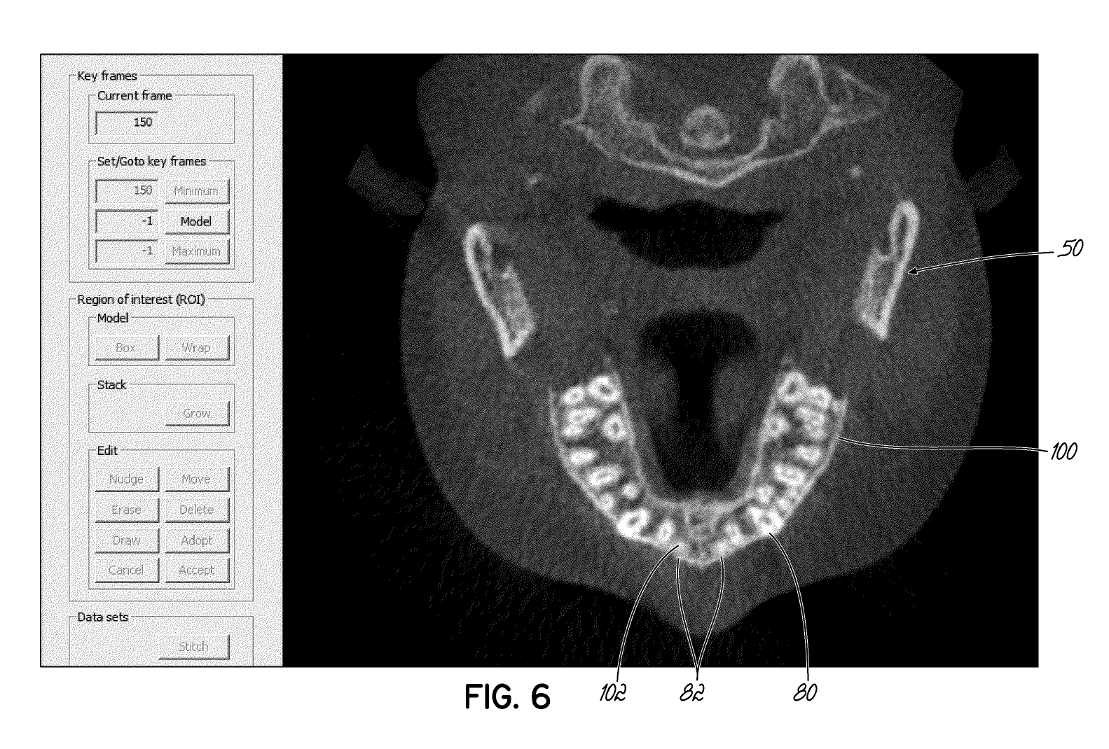

With reference to FIGS. 6, 7, and 8, in one embodiment, the volumetric data captured by the CBCT imaging system 40 may be selectively reduced to only portions of the patient's jaws necessary for evaluating orthodontic treatment options. As is known, volumetric imagery (e.g. CBCT imagery) may contain hundreds of slices (may be referred to as frames herein), each slice including a plurality of voxels that collectively constitute the volumetric imagery 50. To reduce the computation time associated with registration, described below, the practitioner may selectively reduce the volume of information that forms the entire volumetric imagery 50. Thus, in one embodiment, the practitioner may select portions of the volumetric imagery 50 pertinent to orthodontic treatment of the patient. Selection may include removing portions of the volumetric imagery 50 that do not include information associated with the patient's teeth. In doing so, the practitioner may form a modified volumetric imagery 98. It will be appreciated that the modified volumetric imagery 98 may consume less computer resources during manipulation, in view of the reduced data relative to the volumetric imagery 50.

To that end, and with reference to FIG. 6, a practitioner may locate boundaries of information regarding one or more of the patient's teeth in the volumetric imagery 50. By way of example, an upper boundary of an incisor on the maxillary jaw 80 may be located. As shown in FIG. 6, a transverse cross section of the volumetric imagery 50 of the maxillary jaw 80 may be located at or near an apical tip 102 of the root of the incisor 82. This slice may be referenced as a minimum slice 100. Additional slices may be identified to establish other boundaries that define the modified volumetric imagery 98.

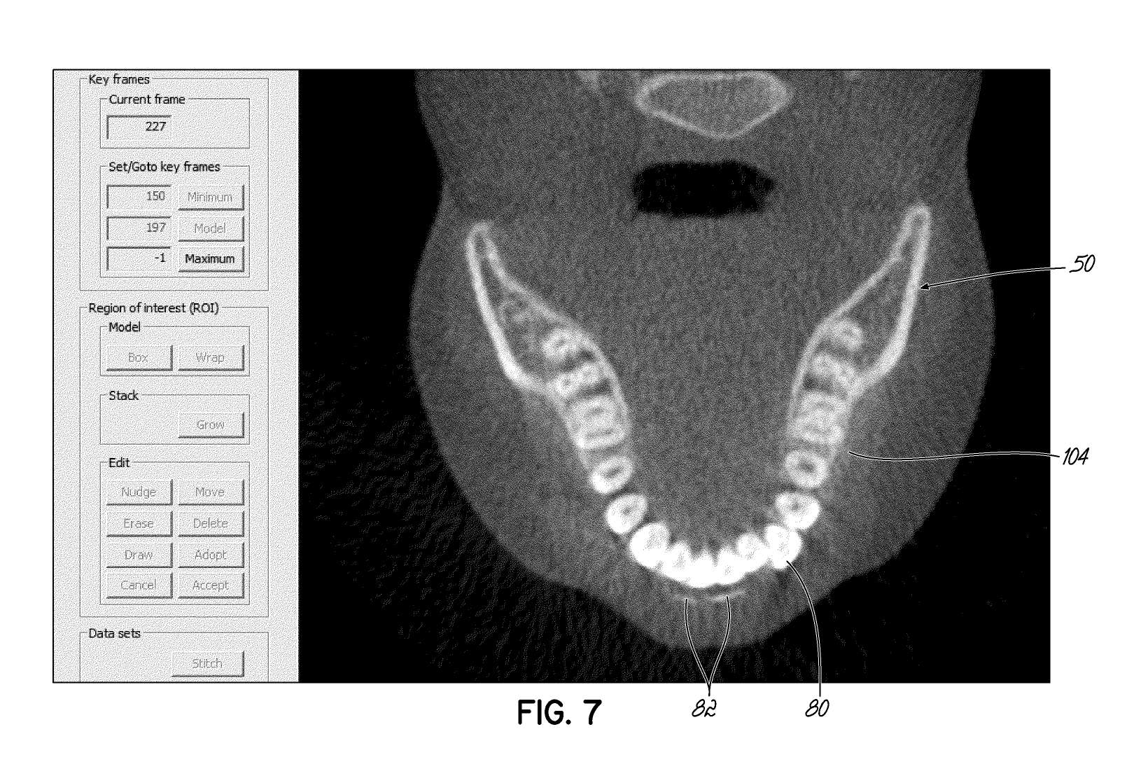

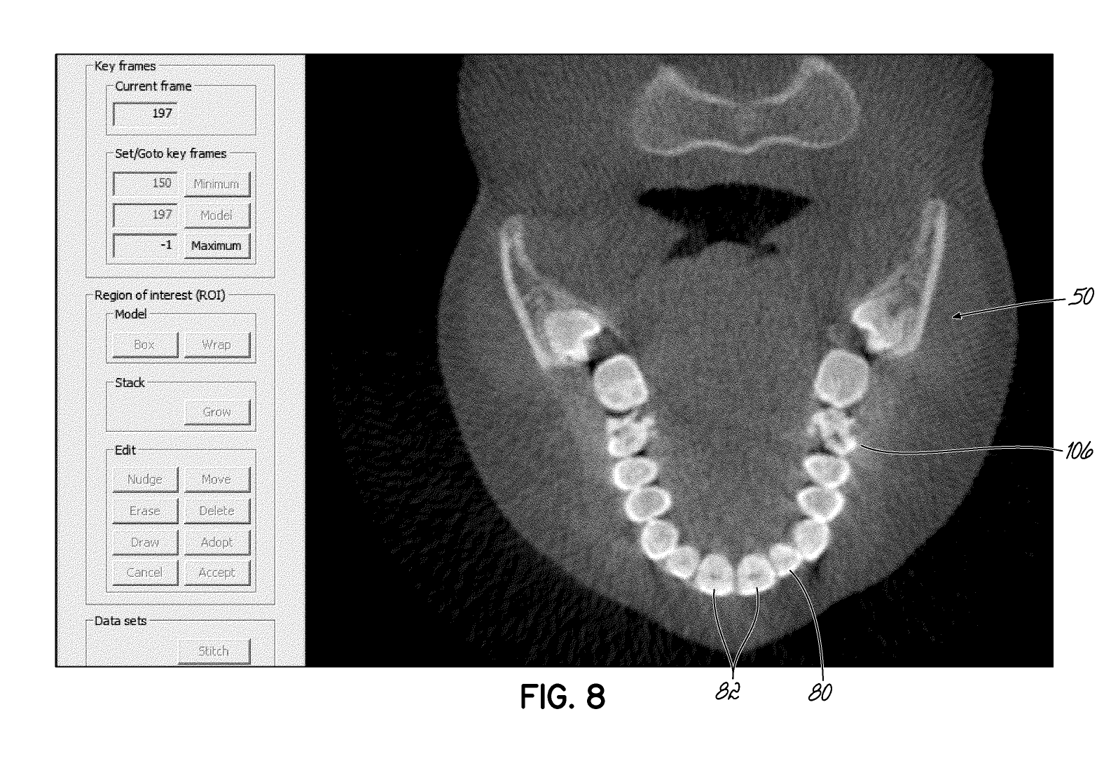

With reference to FIG. 7, an opposing boundary in the volumetric imagery 50 to the minimum slice 100 may be located. In a similar manner as shown in FIG. 6, a transverse slice of the volumetric imagery 50 may locate a maximum slice 104 at or near an incisal margin of the incisor 82. Thus, information regarding the root and the crown of the incisor 82 is captured in a plurality of voxels from the minimum slice 100 to the maximum slice 104. Another slice may be located between the minimum slice 100 and maximum slice 104. For example, and with reference to FIG. 8, another transverse slice may be identified in the volumetric imagery 50 in which the cross section of the selected incisor 82 is largest in diameter. This may be referenced as a model slice 106.

In one embodiment, the modified volumetric imagery 98 may include volumetric data from the minimum slice 100 to the maximum slice 104. Any volumetric data external to the minimum slice 100 and the maximum slice 104 is discarded. In this respect, volumetric data not pertinent to the patient's orthodontic treatment is removed. While containing less data than the volumetric imagery 50, the modified volumetric imagery 98 facilitates more efficient manipulation of the volumetric data pertinent to the patient's teeth.

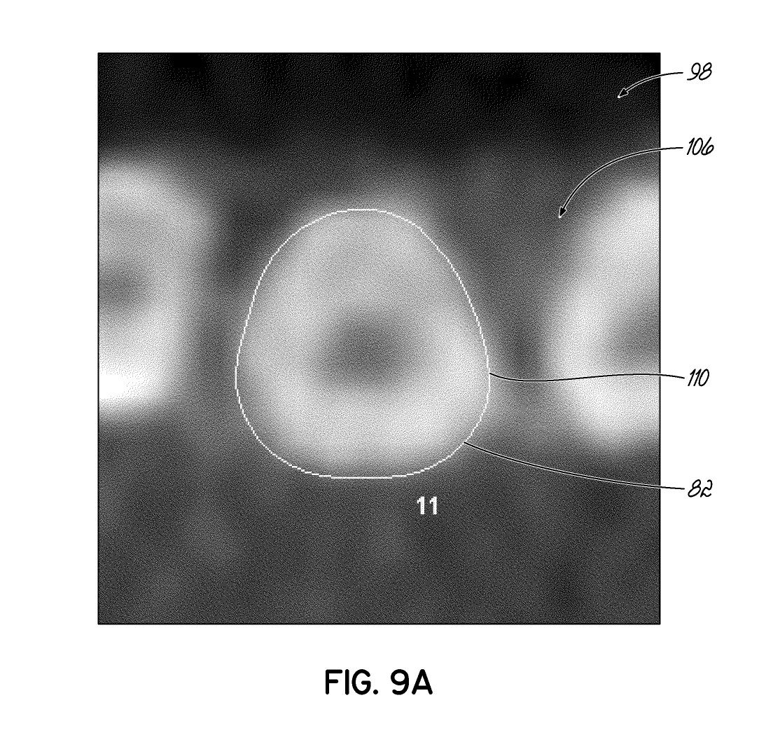

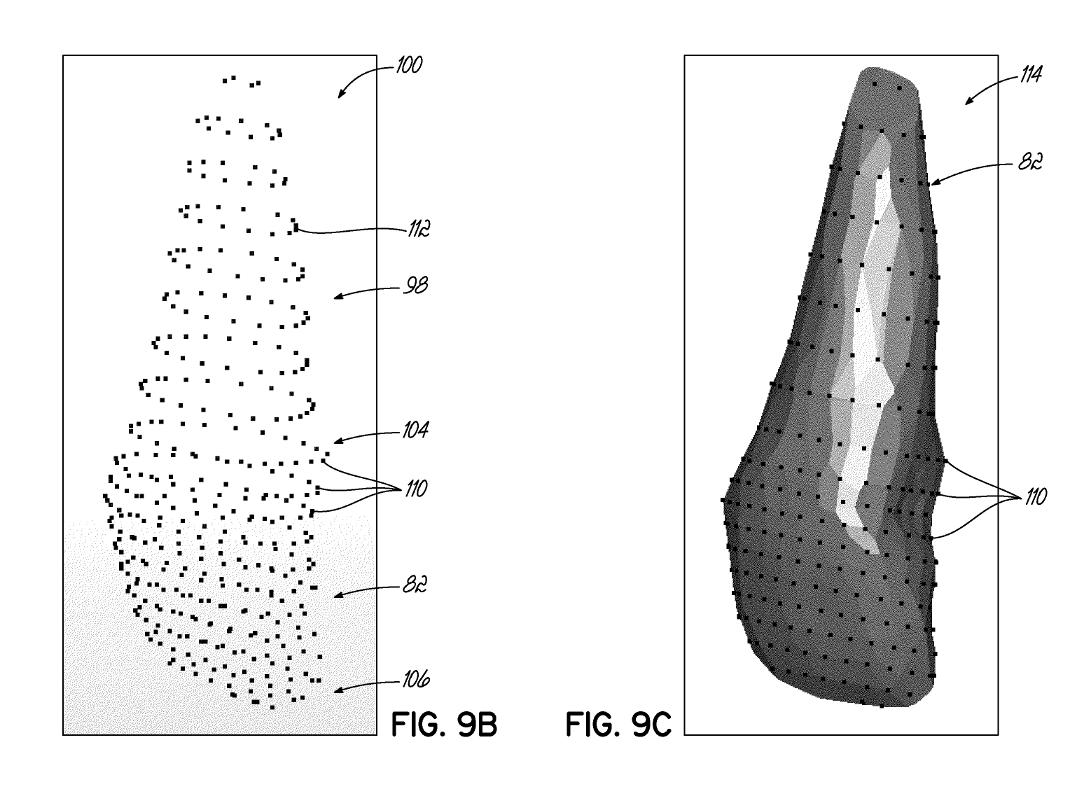

In one embodiment, and with reference to FIGS. 9A, 9B, and 9C, the practitioner may optionally select the model slice 106 for further manipulation. This manipulation may include using an algorithm to find a peripheral edge 110 of the incisor 82 in the model slice 106. Possible algorithms include level set methods described in, for example, an article C. Li et al. “Distance Regularized Level Set Evolution and Its Application to Image Segmentation,” IEEE TRANSACTIONS ON IMAGE PROCESSING, Vol. 19, No. 12, December 2010, which is incorporated by reference herein in its entirety. The practitioner may then propagate additional edges across the intervening slices between the model slice 106 and the minimum slice 100 and between the model slice 106 and the maximum slice 104. As a result of the propagation of the edges 110 of each slice, a point cloud 112 of the incisor 82 may be formed. An exemplary point cloud 112 is shown in FIG. 9B. Interpolating the point cloud 112, forms a 3-D model 114 of the incisor 82 as is shown in FIG. 9C.

The surface imagery 30 may include holes. These are areas of the crown that are not imaged by the imaging system 14. Surface and/or shape data is not available in the holes. The volumetric imagery 50 may be used, such as by the integrating application 52, to fill in at least some data with regard to the crowns at these areas according to commonly owned U.S. Pat. No. 9,135,498, which is incorporated herein in its entirety. It will be appreciated that embodiments of the invention are not limited to any hole filling procedure.

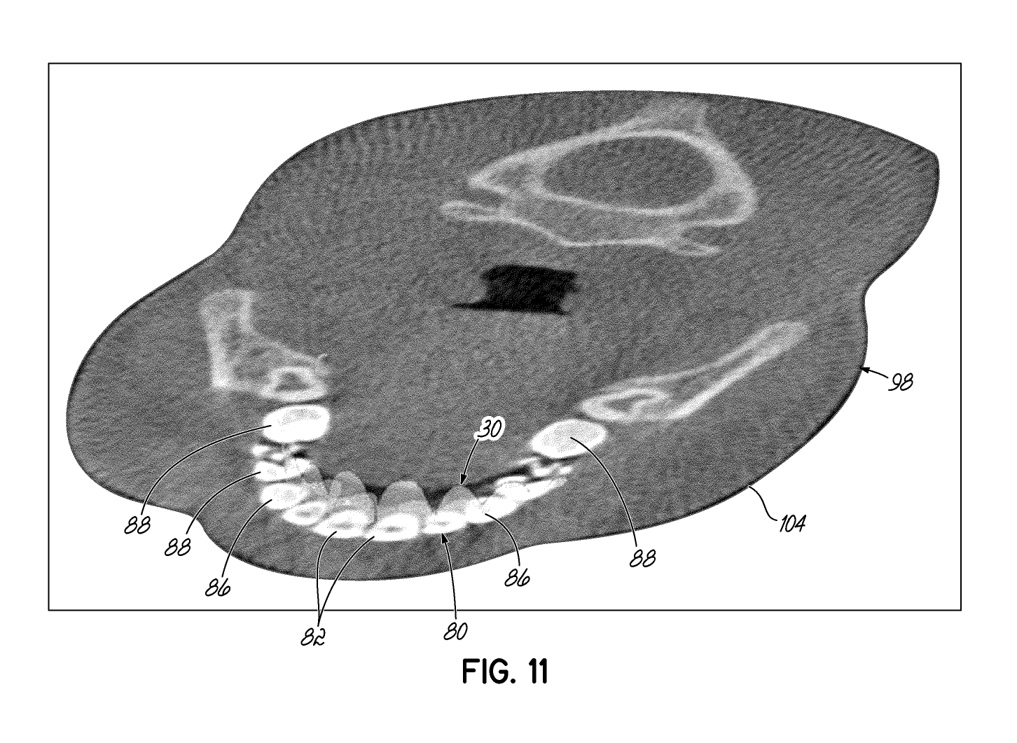

In one embodiment, and with reference to FIG. 10 at 204, following development of the modified volumetric imagery 98, a practitioner may integrate the surface imagery 30 and the modified volumetric imagery 98 (or volumetric imagery 50, if modified imagery is not needed). To that end, the practitioner may bring the surface imagery 30 and the modified volumetric imagery 98 into the same coordinate system, shown in FIG. 11. The two imageries 30 and 98 may overlap in a single virtual space. This may be achieved via the integrating application 52 on the computer 12 within which each of the imageries 30, 98 is received. In one embodiment, the surface imagery 30 and the modified volumetric imagery 98 are the same scale.

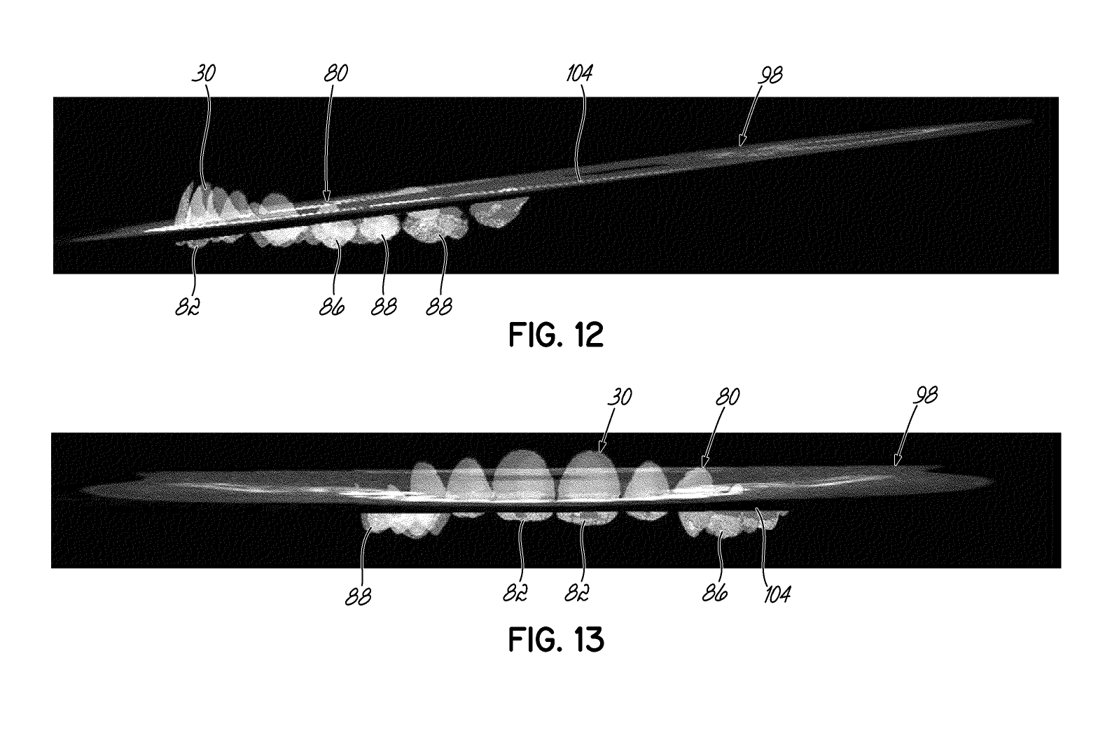

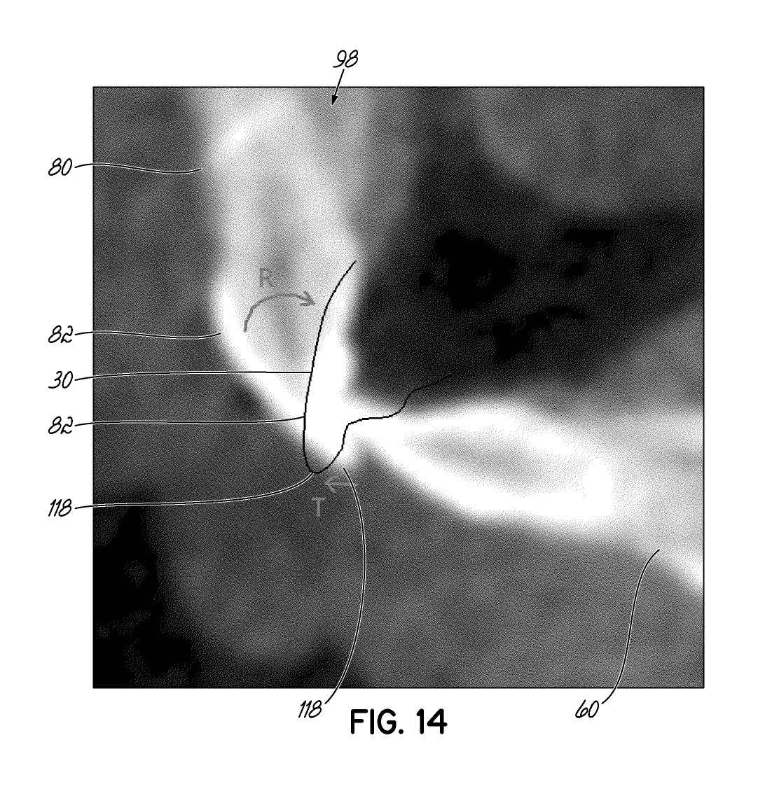

Once the imageries 30 and 98 are in the same coordinate system, the practitioner may roughly register them by manipulating the modified volumetric imagery 98. Rough registration may include positioning the surface imagery 30 and the modified volumetric imagery 98 reasonably close to one another so that the central incisors in each image overlap to a degree. By way of example, the images of the central incisor in each imagery 30, 98 may touch one another. Rough registration generally does not produce exact alignment. Further, while the central incisors may touch, alignment between the two imageries may decrease at locations away from the incisors so that the molars, for example, in each imagery do not touch or are visually out of alignment as is shown best in FIGS. 12 and 13. In other words, the discrepancies between the two imageries 30 and 98 may grow significantly outside of a local neighborhood around the incisors. By way of example only, and with reference to FIGS. 11-13, the practitioner may bring the maximum slice 104 into rough alignment with surface imagery 30. Thus, integrating the imageries 30 and 98 may include visual alignment between a tooth in each of the imageries 30 and 98. Specifically, with reference to FIG. 14, this may include bringing a buccal cusp (BC) tip 118 of the incisor 82 in the surface imagery 30 into rough registration with BC tip 118 of the incisor 82 in the maximum slice 104 in the modified volumetric imagery 98.

While each of the imageries 30 and 98 may include its own coordinate system, in one exemplary embodiment, in the overlapping imageries, the surface imagery 30 establishes a master coordinate system and the modified volumetric imagery 98 is manipulated within that master coordinate system. By way of example only, the coordinate system of the surface imagery 30 may be designated as Ci={Bi|(Xi, Yi, Zi)}, where Bi is a characteristic feature on a selected tooth at the coordinate Xi along an X axis, Yi along a Y axis, and Zi along a Z axis of the coordinate system Ci of the surface imagery 30. The coordinate system of the modified volumetric imagery 98 (or volumetric imagery 50) may be designated as Cv={Bv|(Xv, Yv, Zv)}, where Bv is a characteristic feature on a selected tooth at the coordinate Xv along an X axis, Yv along a Y axis, and Zv along a Z axis of the coordinate system of the modified volumetric imagery 98. Each of the coordinate systems Ci and Cv may be utilized during registration of the surface imagery 30 and the modified volumetric imagery 98. In one embodiment, Ci is the master coordinate system during initial registration and Cv is the slave coordinate system. During later stages of registration, Cv may be the master coordinate system and Ci may be the slave coordinate system.

Alternatively, and according to one embodiment of the invention, the modified volumetric imagery 98 may establish the master coordinate system initially and so the surface imagery 30 may be rotated and translated relative to the fixed orientation of the coordinate system of the modified volumetric imagery 98. The surface imagery 30 may then establish the master coordinate system during later stages of registration.

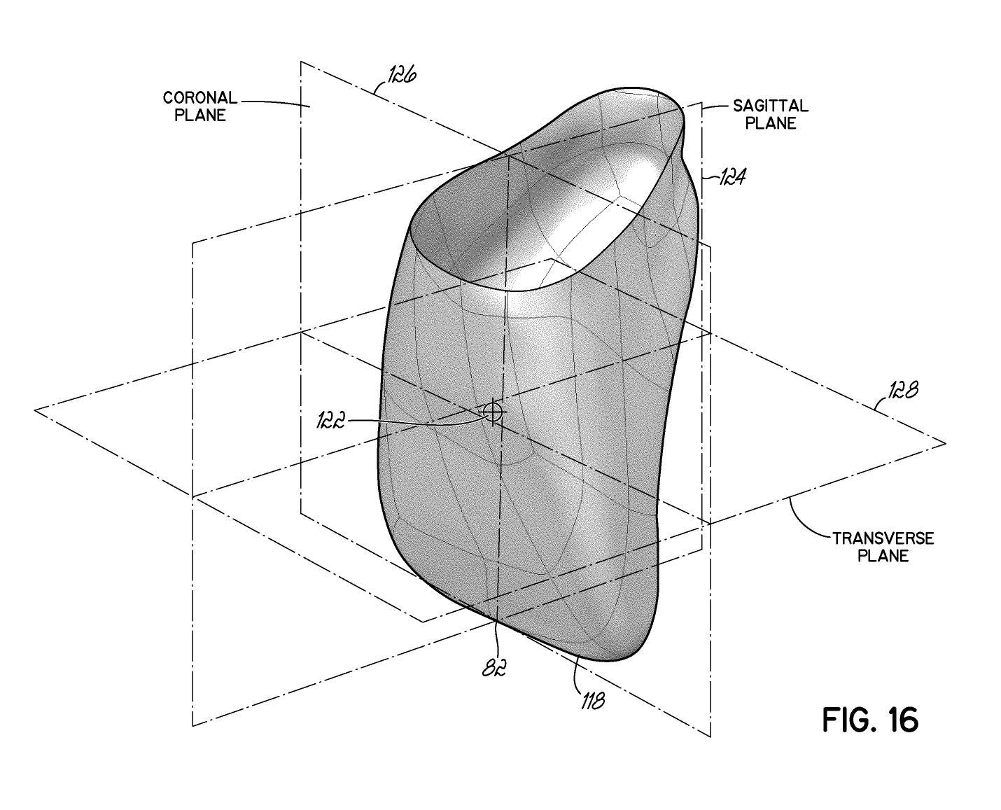

With reference to FIG. 10 at 206, in one embodiment of the invention, once the imageries 30 and 98 are overlaid, one or more planes are prepared through a preselected tooth. In one embodiment, three planes are taken through a preselected tooth at a predetermined location. For example, a first plane, a second plane, and a third plane may be generated at a predetermined location on the surface imagery 30 at one of the maxillary central incisors 82. In one embodiment, the first, second, and third planes are orthogonal to one another. This construction may be generated around each individual tooth. The orientation of the three planes may be dependent on the orientation of the respective tooth. As such, the three planes around one tooth may differ in orientation from another three planes around an adjacent tooth. The three planes may be referred to herein as a sagittal plane, a coronal plane, and a transverse plane. Thus, as referenced herein “sagittal,” “coronal,” and “transverse” are for naming convenience and are not intended to restrict embodiments of the invention to any particular orientation with respect to the patient's body. With that and with reference to FIG. 16, which is described below, the sagittal plane may correspond to the tooth Y-Z plane, a coronal plane may correspond to the tooth X-Z plane, and a transverse plane may correspond to the tooth X-Y plane. In one embodiment, the planes section the preselected tooth at a characteristic feature (e.g., Bi or Bv) further described below.

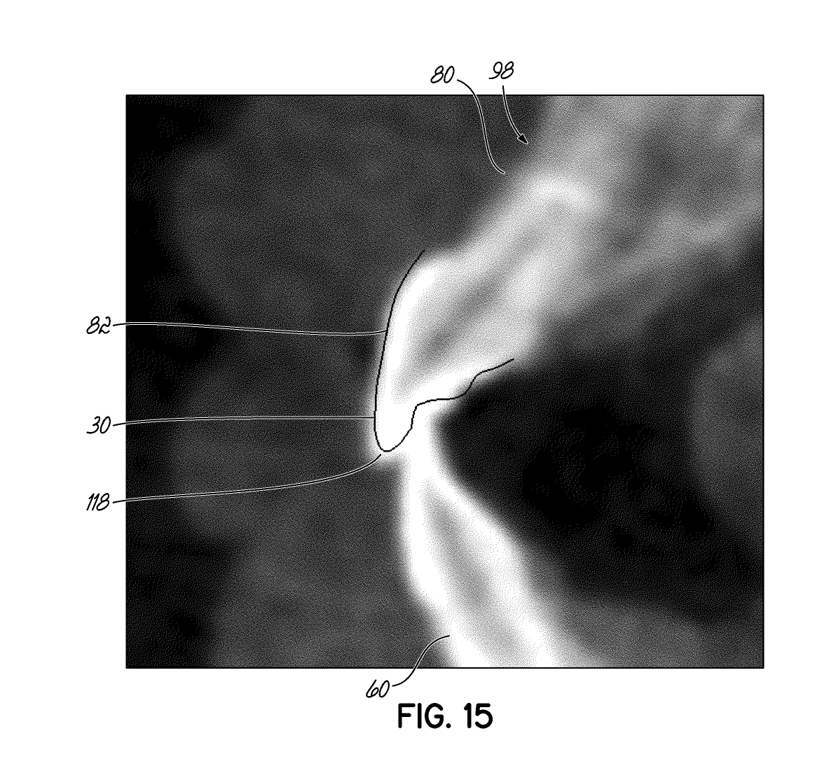

Referring to FIGS. 10 at 206 and 208 and FIGS. 14 and 15, the cross sections in each of the sagittal, coronal, and transverse planes through the selected tooth produce a cross-sectional shape of the tooth in that plane. Thus, for a given tooth, there will be two cross sections, one cross-sectional shape for the surface imagery 30 and one cross-sectional shape for the modified volumetric imagery 98. The cross sections of the tooth in each the surface imagery 30 and the modified volumetric imagery 98 may or may not be the same shape and/or the same size, though they may be similar in both shape and size. Following initial rough alignment as is depicted in FIGS. 11-13 and cross-sectioning as is described above, the practitioner may rotate and/or translate one of the imageries 30, 98 relative to the other in each plane to align the two cross-sectional shapes of the selected tooth.

By alignment, the peripheral outline of the cross sections may be matched so as to be congruent if the cross sections are exactly the same size and shape. Otherwise during alignment there may be deviations between the peripheral outlines of the two cross sections. In this case, the two cross sections may be pattern matched so that they appear to visually align. That is, the outlines of the cross sections may be brought into visual alignment. This type of pattern matching may include moving the slave coordinate system relative to the master coordinate system.

With reference to FIGS. 10 at 210 and 212, registration includes an operation, M, that moves the slave coordinate system (i.e., Ci or Cv, whichever is designated as the slave) relative to the master coordinate system (Cv or Ci, whichever is designated as the master) to match the cross sections of the tooth in the selected plane. This process may be referred to as a coordinate transformation. The operation M may be generally represented by the relation M (Cmaster, Cslave): {Cslave->Cmaster}, in which M represents movement of the slave coordinate system (Cslave) relative to the master coordinate system (Cmaster). Movement may include rotation and/or translation of Cslave relative to Cmaster about Bv and/or about Bi to align cross sections of the tooth in each of imageries 30 and 98.

In accordance with the description above, Ci may be the master coordinate system (Cmaster) during an initial stage of registration and Cv may be the slave coordinate system (Cslave) that is moved relative to Ci according to M (Ci, Cv): {Cv->Ci}. As described above, Ci and Cv may alternate as the master coordinate system. For example, Ci may be the master coordinate system when imagery of the maxillary jaw 80 is registered and Cv may be the master coordinate system when the imagery of the mandibular jaw 60 is registered.

When multiple planes are considered in reference to FIG. 10 at 212, M may be separately conducted in each of a transverse plane, a coronal plane, and a sagittal plane as represented by Mt, Mc, and Ms, respectively, described below. Rotation and translation to pattern match cross sections on a plane-by-plane basis may be generally represented by the following pseudo code:

M (Cmaster, Cslave)
{

while (error > termination threshold)
{
Ms (Cmaster, Cslave);
Mt (Cmaster, Cslave);
Mc (Cmaster, Cslave)
}

{

where, in one embodiment, the error is measured by a difference in running averages of the magnitudes of M in each plane. In other words, as the iteration of M (Cmaster, Cslave) progresses, the amount of M in each plane may be reduced. In this way, the cross section in the slave coordinate system converges on the cross section in the master coordinate system. When the iteration of M (Cmaster, Cslave) reaches a point where the correction produced by a consecutive iteration is insignificant, the cross section in the slave coordinate system is considered to be registered onto the master coordinate system, and the process is stopped. If both the mandibular arch 60 and the maxillary arch 80 are registered, the general representation may include M (Ci, Cv): {Cv->Ci}| first arch and M (Cv, Ci): {Ci->Cv}| second arch, where the first arch includes one of the arches 60 and 80 and the second arch includes the other of the arches 60 and 80. In one embodiment, the first arch and the second arch are registered separately.

Specifically as the general representations described above apply to an exemplary embodiment and with regard to FIGS. 11, 12, and 13, the maximum slice 104 of the modified volumetric imagery 98 is shown overlaid in an initial rough registration within the master coordinate system defined by the surface imagery 30. A cross section of the incisor 82 through each of the modified volumetric imagery 98 and the surface imagery 30 in a sagittal plane, as roughly registered in FIG. 11, is shown in FIG. 14. Cross-sectioning the imageries 30, 98 may be a feature that is available in the integrating application 52.

According to Ms (Ci, Cv): {Cv->Ci}| first arch, in which Ci defines the master coordinate system and the first arch is the maxillary arch 80, the modified volumetric imagery 98 is moved to orient the cross section of the crown of the incisor 82 into alignment with the crown of the incisor 82 (shown as a black profile in FIGS. 14 and 15) in the surface imagery 30. This may include alignment of a characteristic feature in the cross section of the incisor 82 that is visible in both of the surface imagery 30 and the modified volumetric imagery 98, such as with respect to the BC tip 118. The operation Ms may include a rotation R and/or translation T in the sagittal plane, as is shown by corresponding arrows in FIG. 14. Thus, Ms may align or at least begin alignment of the entirety of each of the surface imagery 30 and the modified volumetric imagery 98 by pattern matching of cross sections of the incisor 82 taken in the sagittal plane.

In that regard, the practitioner may utilize pattern recognition techniques to bring about alignment of the surface imagery 30 and the modified volumetric imagery 98 in the sagittal plane of the maxillary arch 80. Pattern recognition techniques mimic human perception. In that regard, in embodiments of the invention in which the practitioner is able to discern the teeth in the modified volumetric imagery 98, pattern recognition algorithms may be used to accurately align the imageries 30, 98. By finding the precise location and orientation of the cross section of the tooth in the modified volumetric imagery 98 relative to the cross section of the tooth in the surface imagery 30, it is possible to obtain deterministic information on how to correct the misalignment between surface imagery 30 and modified volumetric imagery 98.

As a result of rotation R and/or translation T, the modified volumetric imagery 98 is moved relative to the surface imagery 30 so as to align the cross section of the incisor 82 in the modified volumetric imagery 98 with the corresponding cross section of the incisor 82 in the surface imagery 30. This is shown in FIG. 15 in which the cross sections are aligned in accordance with one embodiment of the invention. In one embodiment, the tip 118 of the incisor 82 is selected as a point about which to align the surface imagery 30 with the modified volumetric imagery 98. The central incisors have the simplest shape and largest flat surfaces among all human teeth and so provide an ideal target for pattern matching. However, while a central incisor is shown and described herein to define Bi and Bv (e.g., the tip 118), other anterior teeth (e.g., lateral incisors, canines) may be utilized in a similar fashion if they are distant from distortion causing fillings 94 (see, e.g., FIG. 3) or other similar foreign objects and have easily identifiable features.

Further, other characteristic features on the selected tooth may define Bi and Bv. For example, points determined by measuring one or more dimensions on the selected tooth may be used. By way of specific examples, a centroid of the selected tooth crown, a midpoint, or a most prominent point on the exterior surface of the tooth may be used. The midpoint may be located by identifying a facial axis of the clinical crown (FACC line) for the selected tooth and measuring one half the height of the crown (generally defined from the gingival margin to the incisal edge) on the FACC line. In one embodiment, and with reference to FIG. 16, a centroid 122 is calculated for the crown of the incisor 82. Once the centroid 122 is identified, a sagittal plane 124, a coronal plane 126, and a transverse plane 128 are constructed through the centroid 122. The planes 124, 126, and 128 may then be used to define cross sections of the tooth in each of the imageries 30 and 98.

There may remain a slight misalignment between the surface imagery 30 and the modified volumetric imagery 98 following an initial movement Ms. This may be addressed by additional movements Mt and/or Mc. In that regard and with reference to FIG. 14, the practitioner may further rotate and translate the modified volumetric imagery 98 in the sagittal plane according to Ms by observing other teeth, such as an overlap between the molars 68 in the surface imagery 30 and the molars in the modified volumetric imagery 98 in cases in which the patient does not have fillings at this location. Thus, additional movements at locations distant from Bv, Bi may improve alignment.

As is described below, once the cross sections are aligned in the sagittal plane, the practitioner may proceed to the next plane, such as coronal plane 126 or transverse plane 128 (FIG. 16), and match the profiles of the two cross sections in that plane. Thus, Mc or Mt may follow Ms, described above with reference to FIGS. 14 and 15. A similar procedure may be taken with the third plane for whichever Mc and Mt remains to be completed. In this way, the imageries 30 and 98 are registered on a plane-by-plane basis by a pattern matching process according to Ms, Mc, and Mt, with respect to Bi, Bv in which the cross sections of the selected tooth in the selected plane are aligned. Alignment may include visually perceptible alignment or alignment as determined algorithmically.

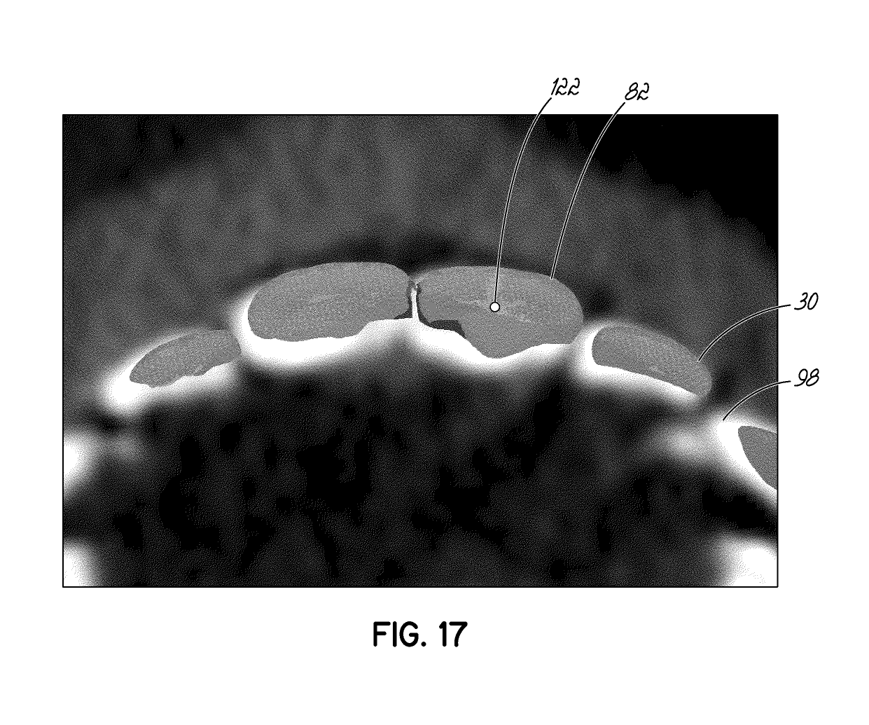

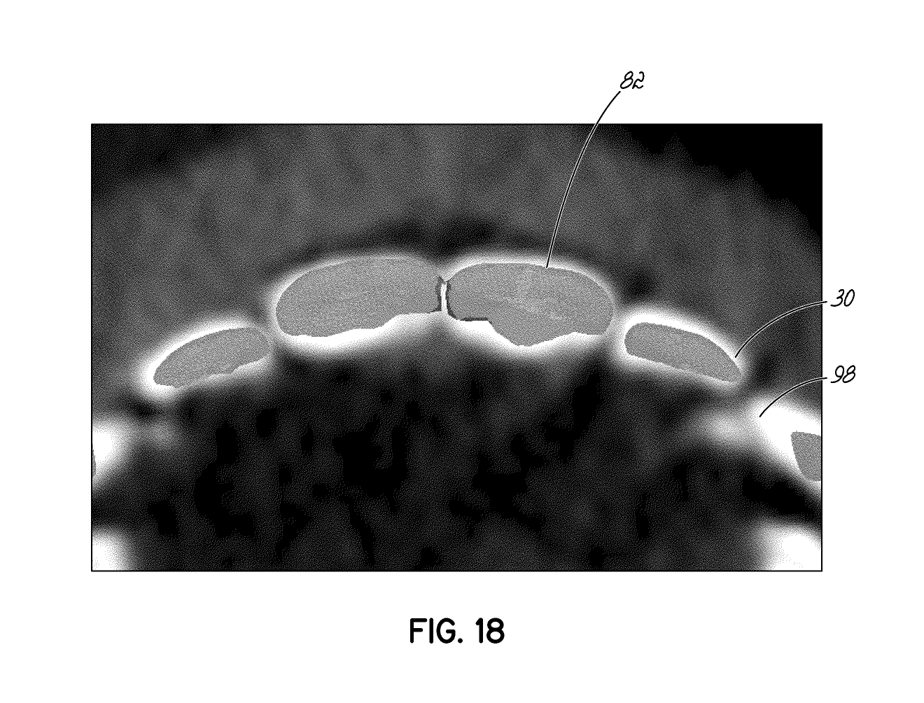

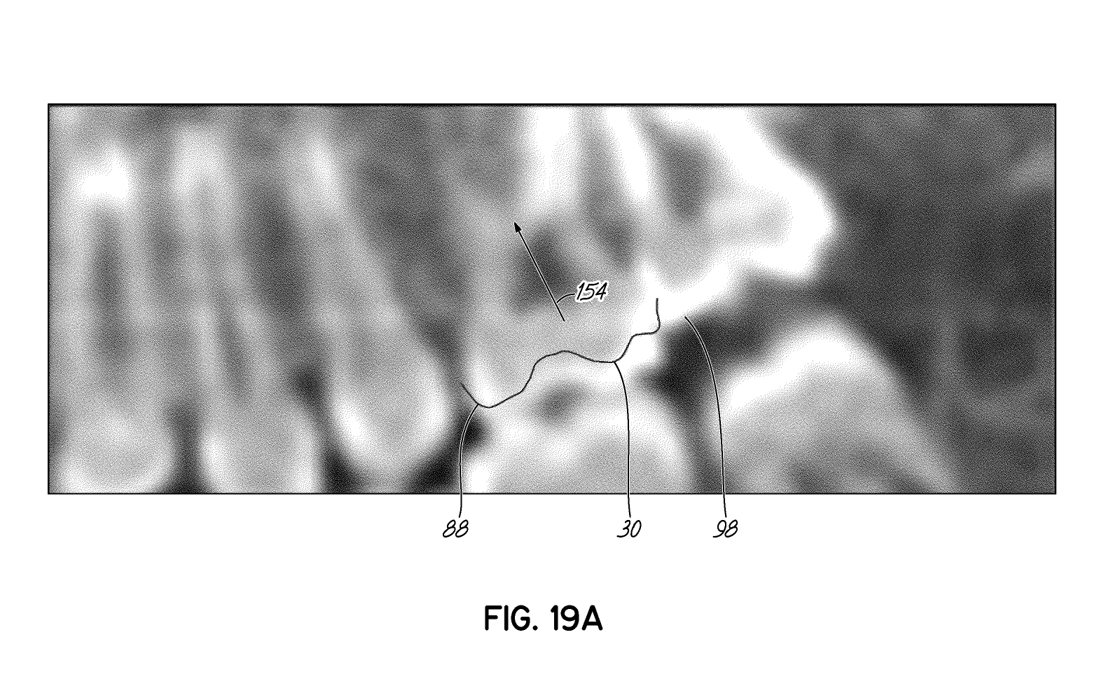

By way of further example, and with reference to FIG. 10 at 212 and FIGS. 17 and 18, registration according to Mt (Ci, Cv): {Cv->Ci}| maxillary arch is shown. According to Mt, the practitioner may cross section the surface imagery 30 in a transverse plane, such as at the centroid 122 of the incisor 82. The operation, Mt, may then include rotation and translation of the modified volumetric imagery 98 to align the cross section of the incisor 82 of modified volumetric imagery 98 with the cross section of the incisor 82 of surface imagery 30 in the transverse plane, again using pattern recognition techniques. The result of Mt is shown in FIG. 18. As shown, by Mt, the practitioner may further align the modified volumetric imagery 98 with the surface imagery 30. Although not shown, rotation and/or translation in the coronal plane according to Mc (Ci, Cv): {Cv->Ci}| maxillary arch may complete an initial round of M (Cmaster, Cslave): {Cslave->Cmaster}| maxillary arch. Mc may include rotation and translation in the coronal plane 126. By Mc, the cross section of the incisor 82 of modified volumetric imagery 98 may be aligned with the cross section of the incisor 82 of surface imagery 30 in a coronal plane.

Each of Ms, Mt, and Mc may be completed in a series and the error calculated and checked against the termination threshold for that series. If the error is greater than the termination threshold, each of Ms, Mt, and Mc may be repeated. So, the registration process may be repeated until the calculated error is less than the termination threshold. In this way, any misalignment between the cross sections of the tooth in the surface imagery 30 and in the modified volumetric imagery 98 is corrected in each plane independently for the maxillary arch. Advantageously, this greatly reduces complexity of alignment of the coordinate systems, because embodiments of the invention eliminate the necessity of evaluating simultaneous movement across multiple planes. The order of operations Ms, Mt, and Mc described above is not limiting. The order of each of the movements Ms, Mt, and Mc may be different from that shown and may vary even between the first iteration and last iteration of the overall operation M (Cmaster, Cslave)|maxillary arch. When the error in alignment is reduced to less than the termination threshold, the registration of the modified volumetric imagery 98 to the surface imagery 30 for the patient's maxillary arch 80 may be terminated. This leaves the imageries 30 and 98 of the mandibular arch 60 to be registered, if necessary.

In that regard, according to FIG. 10 at 214, in one embodiment, registration may be conducted separately for each of the mandibular arch 60 and maxillary arch 80 according to the following: M (Ci, Cv): {Cv->Ci}| first arch and M (Cv, Ci): {Ci->Cv}| second arch, where the first arch is one of the mandibular arch 60 or maxillary arch 80 and the second arch is the other of the maxillary arch 80 or mandibular arch 60. Continuing the above example, registration of the surface imagery 30 and the modified volumetric imagery 98 of the mandibular arch 60 may be undertaken following registration of maxillary arch 80.

In one embodiment, each of Ms, Mt, and Mc is completed in the sagittal, transverse, and coronal planes of the mandibular arch 60, respectively, in a manner similar to that described above with regard to the maxillary arch 80. In particular, registration of the mandibular arch 60 according to M (Cv, Ci): {Ci->Cv}| mandibular arch may include:

Ms (Cmaster, Cslave)|mandibular arch;

Mt (Cmaster, Cslave)|mandibular arch; and

Mc (Cmaster, Cslave)|mandibular arch, in which Ms describes movements in a sagittal plane, Mt describes movements in a transverse plane, and Mc describes movements in a coronal plane similar to Ms, Mt, and Mc described above with regard to the maxillary arch 80.

In one embodiment, Cslave as a result of M (Cmaster, Cslave):{Cslave->Cmaster}| first arch, when the calculated error is less than the termination threshold, becomes Cmaster for the second arch in M (Cmaster, Cslave):{Cslave->Cmaster}| second arch. In an exemplary embodiment described above with reference to FIGS. 14-18, Ci is the master coordinate system. During M (Ci, Cv)| maxillary arch, a new relative position of Cv is created as the modified volumetric imagery 98 is moved relative to the surface imagery 30. In one embodiment, the new Cv from that movement is then utilized in a subsequent operation M for the second arch. More particularly, the new position for the modified volumetric imagery 98 (i.e., a new Cv) following registration according to M (Ci, Cv): {Cv->Ci}| maxillary arch, described above, may be utilized as the master coordinate system for the mandibular arch 60 during M (Cv, Ci): {Ci->Cv}| mandibular arch. Accordingly, each of Ms, Mt, and Mc for the mandibular arch is relative to the new master coordinate system.

Switching the master coordinate systems to the modified volumetric imagery 98 when proceeding with M (Cmaster, Cslave)| mandibular arch assumes that the occlusion or bite conditions of the patient during the CBCT scan is a good model to follow and that the occlusion of the intra-oral imagery (i.e., surface imagery 30) should be slightly modified to match the modified volumetric imagery 98. It will be appreciated that the imageries of the mandibular arch and maxillary arch in the surface imagery 30 may be taken separately because the patient's mouth is open or the impressions are of separate arches. Further in that regard, the two imageries 30 and 98 should be taken within a short time of one another, such as in a single office visit. This timing ensures that the teeth 62, 64, 66, and 68 in the mandibular arch 60 or the teeth 82, 84, 86, 88 in the maxillary arch 80 have not moved relative to one another within the gingiva 70 between development of surface imagery 30 and volumetric imagery 50. At 216 in FIG. 10, once the imageries 30 and 98 are aligned, a 3-D model may be prepared such that the practitioner may more accurately prepare a treatment plan, design appliances, and/or prepare a post-treatment evaluation of the patient.

The registration process of M (Ci, Cv): {Cv->Ci}| maxillary arch and then M (Cv, Ci): {Ci->Cv}| mandibular arch may be repeated either with respect to one arch and then the other arch or M may be repeated within a single arch multiple times. Alternatively, a combination of the above may be completed. By way of example, registration may include 3 or 4 complete rounds of M (Ci, Cv)| first arch followed by 3 or 4 complete rounds of M (Cv, Ci)| second arch. Advantageously, embodiments of the invention do not rely on fitting or thresholding schemes. The resulting alignment of the cross sections of the selected tooth on each of the surface imagery 30 and the modified volumetric imagery 98 is not an approximation and is not sensitive to poor density distribution across the modified volumetric imagery 98 due to low x-ray dosage or for other reasons. It will be appreciated that the volumetric imagery 50 (i.e., the unmodified volumetric imagery) may be utilized instead of the modified volumetric imagery 98. While registration is described with regard to the practitioner visually manipulating the surface imagery 30 and the modified volumetric imagery 98 via the integrating application 52, for example, manipulation may also be performed automatically via an algorithm on the computer 12.

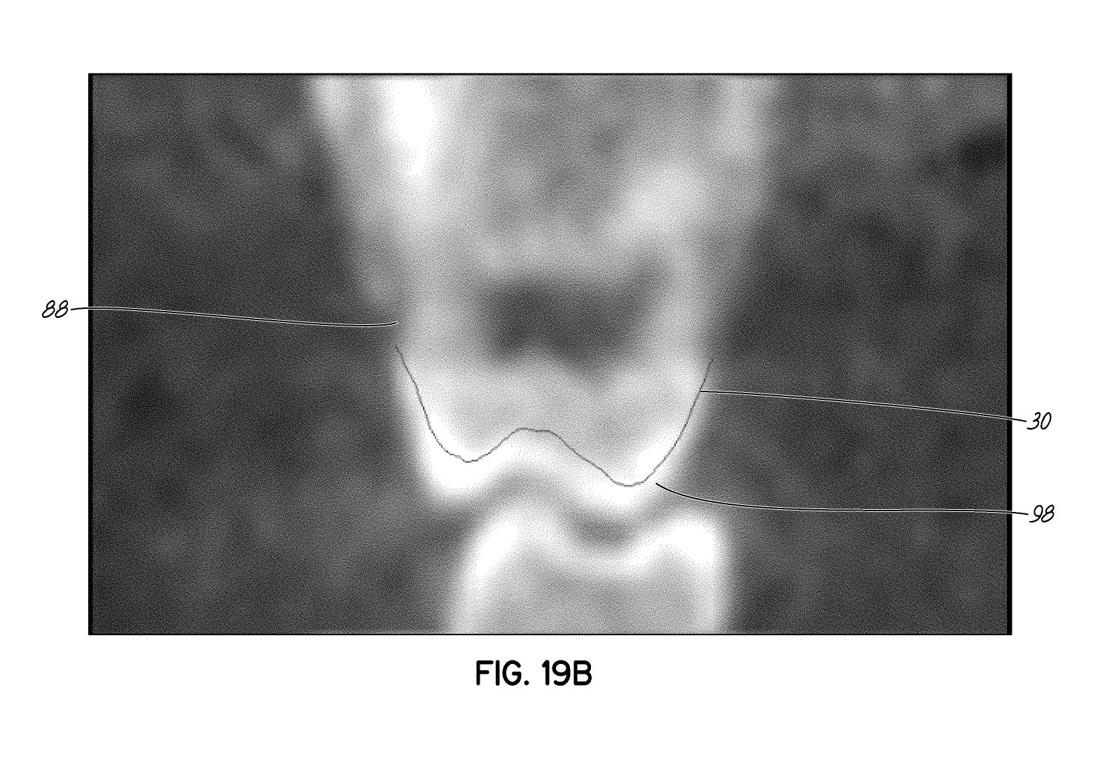

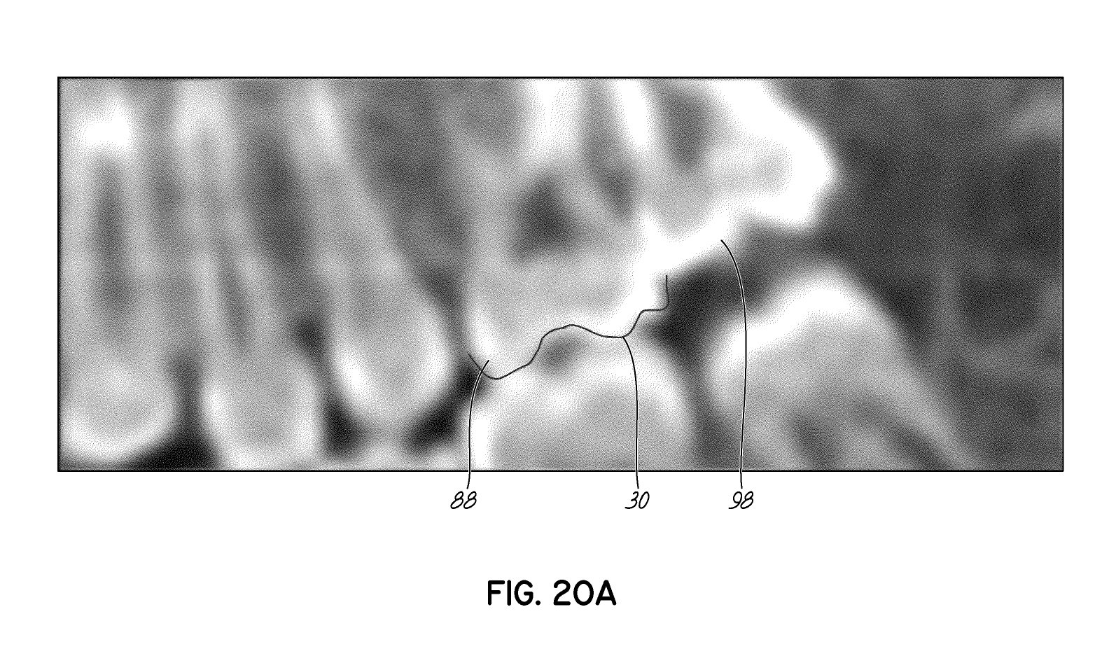

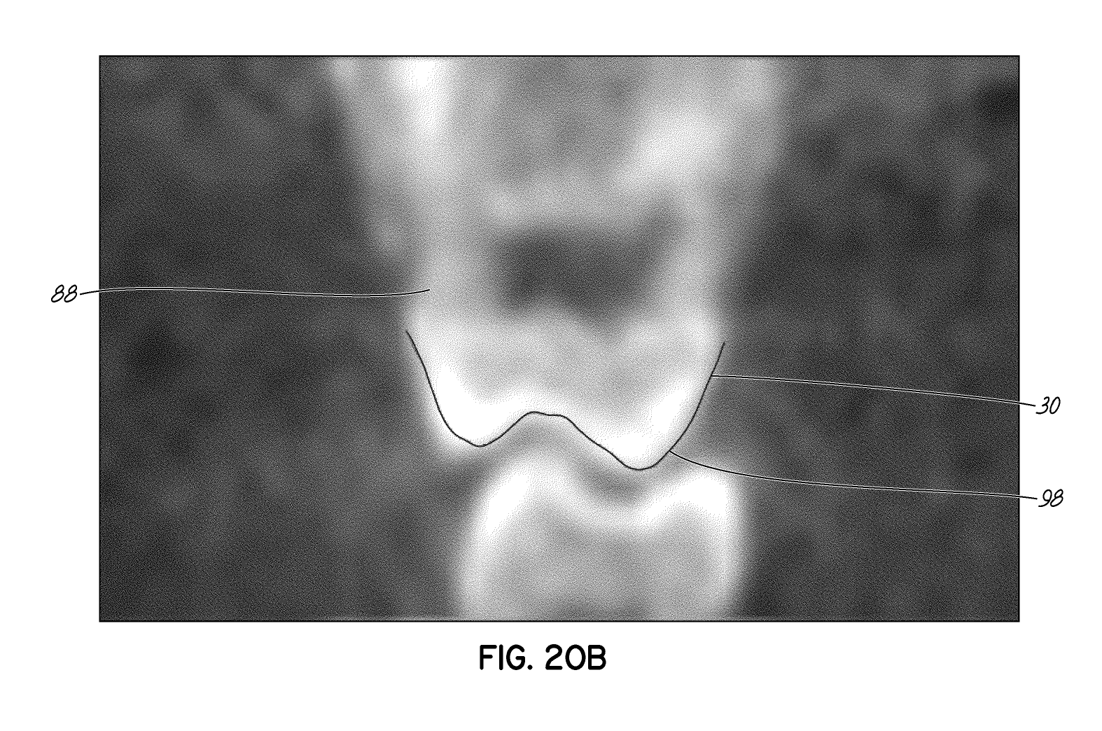

Advantageously, movement M in one plane, such as movement in one of the sagittal, transverse, or coronal planes, may reduce error between imageries 30 and 98 in one or both of the other two planes. This is shown by way of example with reference to FIGS. 19A, 19B, 20A, 20B, 21A, 21B, 22A, and 22B. Registration according to Mc in the coronal plane for the molar 88 is shown by the arrow 154 in FIG. 19A. Movement in the coronal plane according to Mc may reduce error in the sagittal plane shown in FIG. 19B. After Mc, the error between the surface imagery 30 and the modified volumetric imagery 98 at the molar 88 is shown in FIGS. 20A and 20B. As shown, the error between the surface imagery 30 and the modified volumetric imagery 98 of the molar 88 in each of the coronal and sagittal planes is also reduced by comparison of the relative positions of the imageries 30, 98 in FIGS. 19A and 19B with the corresponding imageries 30, 98 in 20A and 20B. A reduction in the error in one plane may also improve alignment in each of the other planes. This may occur even in the absence of separate operation Mx in the other plane. Although not shown, the error between the surface imagery 30 and the modified volumetric imagery 98 of the molar 88 in the transverse plane may also be reduced by a movement in one of Ms or Mc.

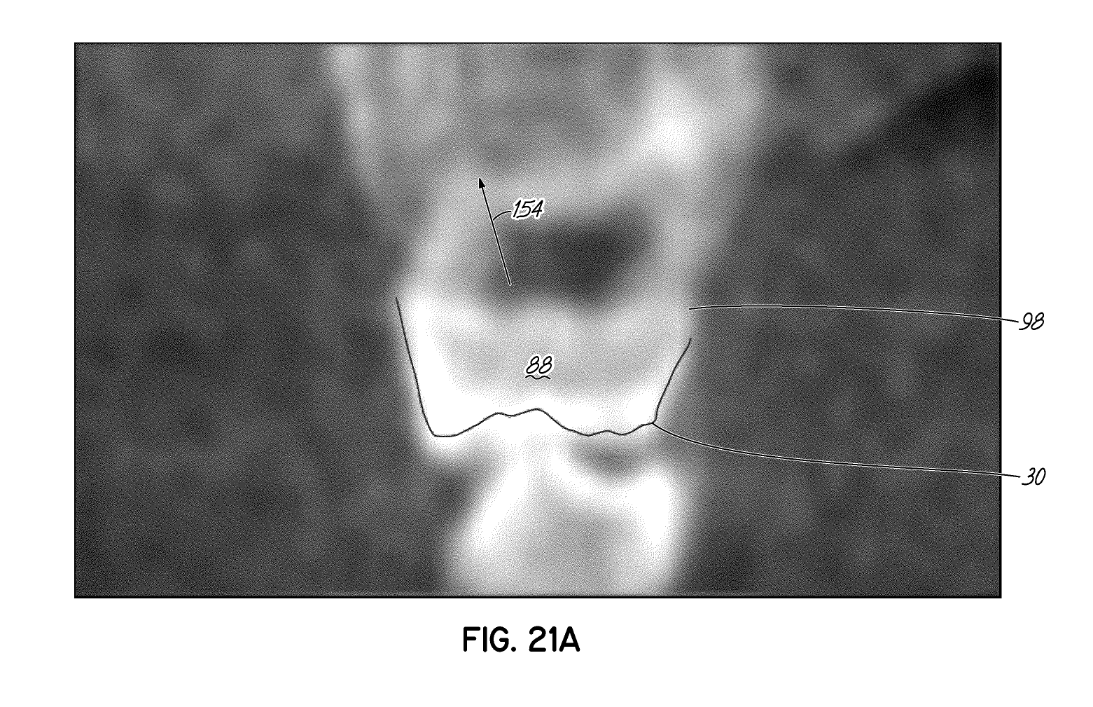

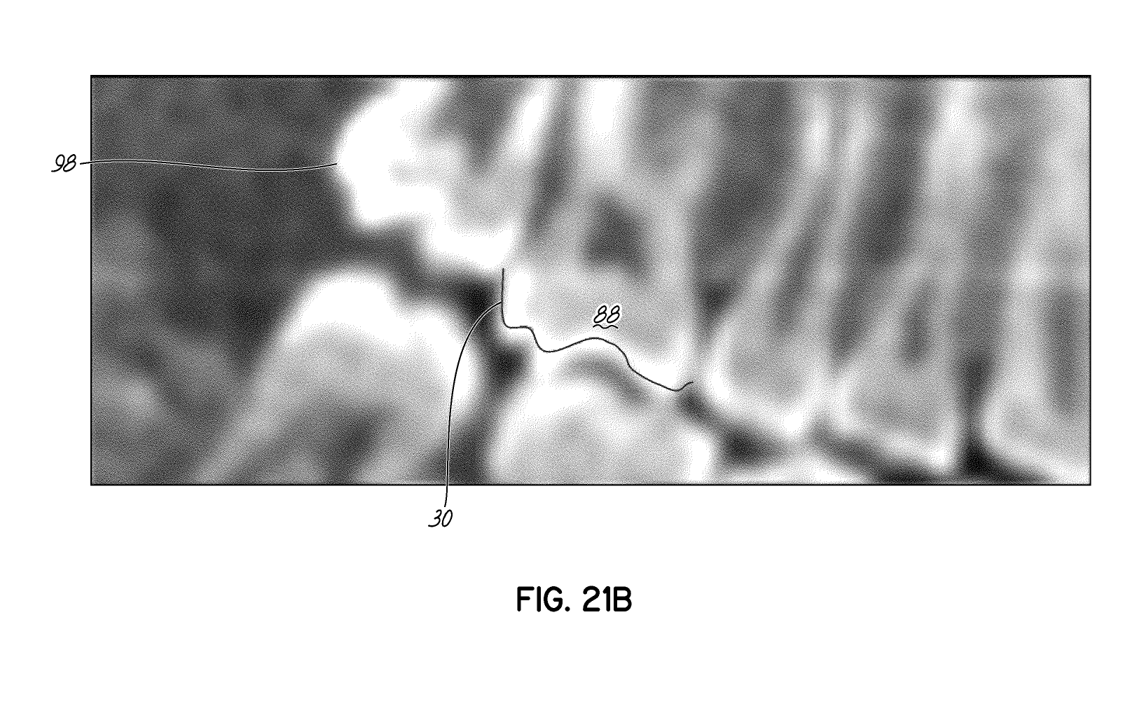

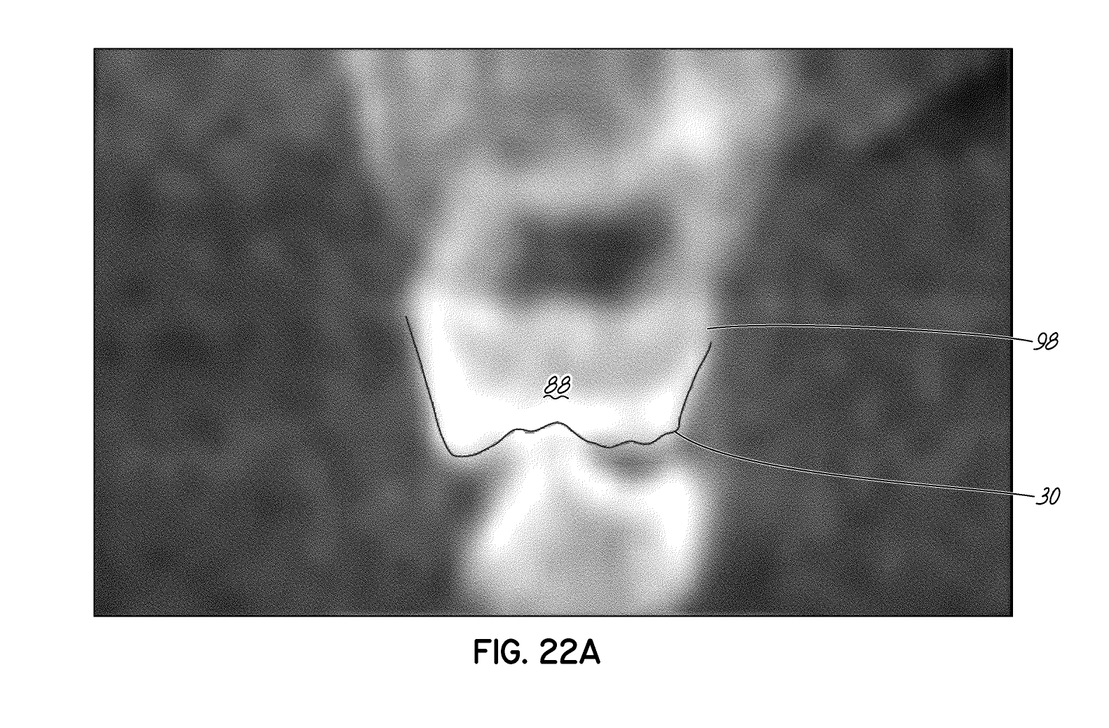

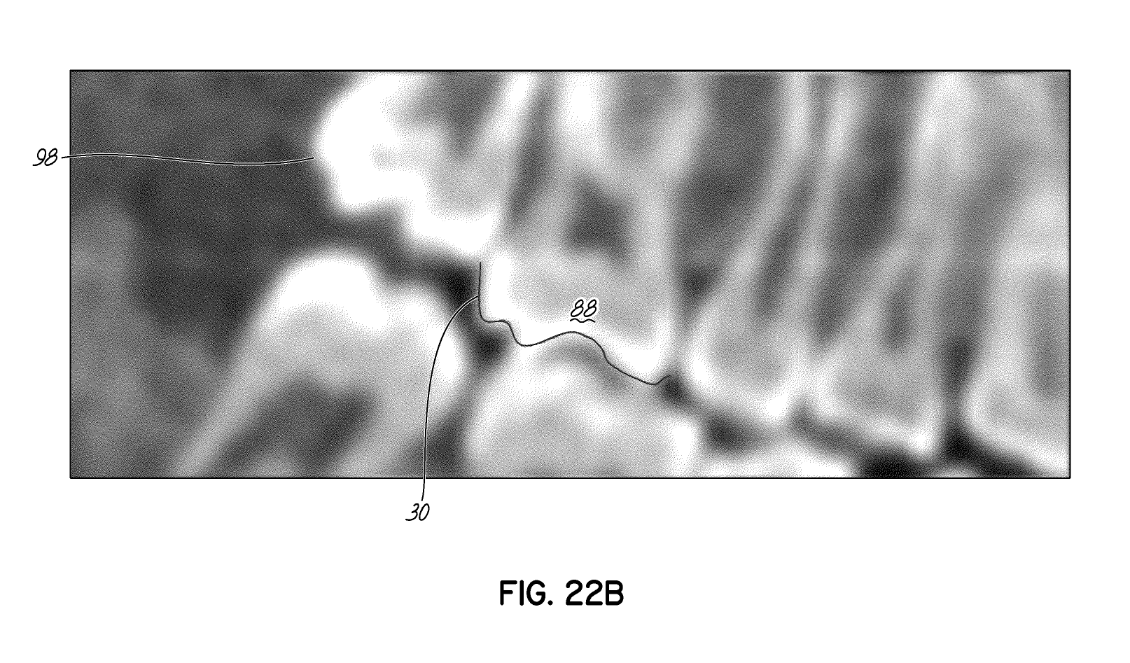

By way of additional example and with reference to FIGS. 21A, 21B, 22A, and 22B, registration according to Ms in the sagittal plane for the molar 88 is shown by arrow 154 in FIG. 21A. After Ms, the error between the surface imagery 30 and the modified volumetric imagery 98 in the molar 88 is shown in FIGS. 22A and 22B. Again, the error between the surface imagery 30 and the modified volumetric imagery 98 of the molar 88 in the coronal plane (FIG. 22B) is reduced even in the absence of a separate movement Mc.

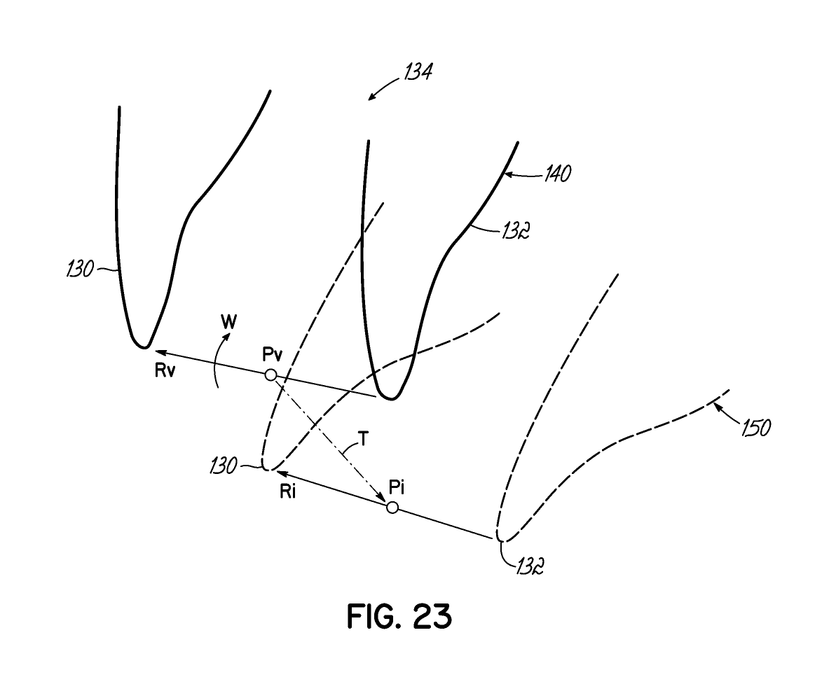

In one embodiment and with reference to FIG. 23, rather than a single characteristic feature of each the surface imagery 30 and the modified volumetric imagery 98 (i.e., Bi and Bv), multiple features may be utilized as reference locations and about which the imageries 30, 98 are aligned. By way of example only, at least two teeth are selected for alignment. Sagittal plane cuts of two central incisors 130, 132 through their BC points are shown in a maxillary arch 134 for a volumetric imagery 140 and for a surface imagery 150.

With regard to M (Cmaster, Cslave)| maxillary arch, above, registration of the imagery 140 to the imagery 150 in the sagittal plane according to Ms may include translation and rotation due to the alignment of two fixed length vectors: (1) vector Rv between BC points on two adjacent central incisors 130, 132 in the imagery 140 and (2) vector Ri between BC points on two adjacent central incisors 130, 132 in the imagery 150. In particular, Ms may include translation T and compound rotations W with respect to a midpoint Pv of vector Rv relative to a midpoint Pi of vector Ri. Translation T (represented as a vector in FIG. 23) does not generally lie strictly on a single sagittal plane and instead spans across multiple planes. Once Pv and Pi coincide, additional compound rotations W may be required to further align Rv with Ri. Similarly, W is not generally limited to one plane but spans across multiple planes. In summary, by considering the sagittal plane cross sections of two central incisors together, movement of vector Rv toward Ri may correct misalignments in each of the transverse and coronal planes as well.

While the invention has been illustrated by a description of various embodiments and while these embodiments have been described in considerable detail, it is not the intention of the Applicant to restrict or in any way limit the scope of the appended claims to such detail. Additional advantages and modifications will readily appear to those skilled in the art. The invention in its broader aspects is therefore not limited to the specific details, representative apparatus and method, and illustrative examples shown and described. Accordingly, departures may be made from such details without departing from the spirit or scope of the Applicant's general inventive concept.

What is claimed is:

1. A method for registering a first imagery of a patient's tooth and a second imagery of the patient's tooth comprising:
integrating the first imagery of a tooth and the second imagery of the tooth so that the first imagery is roughly registered with the second imagery;
cross-sectioning the first imagery and the second imagery through the tooth; and
moving the second imagery relative to the first imagery so as to align the cross section of the second imagery with the cross section of the first imagery.

2. The method of claim 1, wherein moving includes at least one of rotating and translating the second imagery relative to the first imagery.

3. The method of claim 2, wherein rotating and translating is relative to a characteristic feature of the tooth in the first imagery.

4. The method of claim 1, wherein the tooth is an anterior tooth.

5. The method of claim 1, wherein the tooth is an incisor.

6. The method of claim 1, further including selecting a characteristic feature on the tooth and wherein cross-sectioning includes cross-sectioning the first imagery at the characteristic feature.

7. The method of claim 6, wherein selecting a characteristic feature of the tooth includes selecting a characteristic feature of an incisor.

8. The method of claim 1, wherein cross-sectioning includes cross-sectioning along at least one of a sagittal plane, a transverse plane, and a coronal plane.

9. The method of claim 8, wherein moving includes at least one of rotating and translating the second imagery on a plane-by-plane basis in each of the sagittal plane, the transverse plane, and the coronal plane.

10. The method of claim 1, wherein after moving the second imagery, the method further includes evaluating an error in the position of the second imagery relative to the first imagery.

11. The method of claim 1, further including repeating moving the second imagery relative to the first imagery to further align the cross section of the second imagery with the cross section of first imagery.

12. The method of claim 1, wherein after moving the second imagery, the method further includes moving the first imagery relative to the second imagery.

13. The method of claim 1, wherein the first imagery is a surface imagery and the second imagery is a volumetric imagery.

14. A method for registering a first imagery of a patient's tooth and a second imagery of the patient's tooth comprising:
integrating the first imagery of a tooth and the second imagery of the tooth so that the first imagery is roughly registered with the second imagery;
selecting a characteristic feature of the tooth in the first imagery; and
moving the second imagery relative to the first imagery about the characteristic feature so as to align the second imagery with the first imagery.

15. The method of claim 14, wherein selecting the characteristic feature includes selecting a feature on an anterior tooth.

16. The method of claim 14, wherein selecting the characteristic feature includes selecting a feature on an incisor.

17. The method of claim 14, further including cross-sectioning the first imagery through at least the characteristic feature.

18. The method of claim 17, wherein cross-sectioning includes cross-sectioning along at least one of a sagittal plane, a transverse plane, and a coronal plane.

19. The method of claim 17, wherein cross-sectioning includes cross-sectioning the second imagery.

20. The method of claim 14, wherein the first imagery is a surface imagery of the tooth and the second imagery is a volumetric imagery of the tooth.

21. The method of claim 14, wherein moving includes at least one of rotating and translating the second imagery.

22. The method of claim 21, wherein after moving the second imagery, the method further includes evaluating an error in a position of the second imagery relative to the first imagery.

23. The method of claim 14, further including repeating moving the second imagery relative to the first imagery.

24. The method of claim 14, wherein after moving the second imagery, the method further includes moving the first imagery relative to the second imagery about the characteristic feature.

25. The method of claim 1, wherein the first imagery is a surface imagery and the second imagery is a volumetric imagery.

26. A dental registration system comprising:
a first imaging system that is capable of capturing information that is usable to prepare a first imagery of a patient's tooth;
a second imaging system that is capable of capturing information that is usable to prepare a second imagery of the patient's tooth, the second imagery being different than the first imagery; and
at least one computer operatively coupled to the first imaging system and the second imaging system and including an integrating application that is capable of manipulating the first imagery and the second imagery,
wherein the integrating application is capable of cross-sectioning the first imagery and the second imagery along at least one of a sagittal plane, a transverse plane, and a coronal plane and is capable of moving the second imagery relative to the first imagery.
